(19) United States
(12) Patent Application Publication
(10) Pub. No.: US 2026/0098305 A1
(43) Pub. Date: Apr. 9, 2026

(54) INFLAMMATORY DISEASE GENE PANEL

(71) Applicant: Ludwig Enterprises, Inc., Sparks, NV (US)

(72) Inventors: Marvin S. Hausman, Chuluota, FL (US); Sapphire Thalia Villafana, Miami, FL (US); Kyle H. Ambert, Portland, OR (US)

(21) Appl. No.: 19/112,995

(22) PCT Filed: Sep. 20, 2023
(86) PCT No.: PCT/US2023/074690

§ 371 Date: Mar. 19, 2025

Related U.S. Application Data
(60) Provisional application No. 63/376,400, filed on Sep. 20, 2022.
(60) Provisional application No. 63/500,901, filed on May 8, 2023.

(51) Int. Cl. C12Q 1/6886; C12Q 1/6883; G01N 33/68

(52) U.S. Cl. CPC C12Q 1/6886; C12Q 1/6883; G01N 33/57515; G01N 33/57535; G01N 33/5754; G01N 33/6893; C12Q 2600/158; G01N 2800/323; G01N 2800/328; G01N 2800/368; G01N 2800/52; G01N 2800/7095

(57) ABSTRACT

Provided is a method of determining whether a patient has bladder cancer, colon cancer, or breast cancer. Also provided is a method of determining whether a patient has a chronic inflammatory disease. Additionally provided is a method of evaluating patient response to a treatment for a chronic inflammatory disease. Further provided is a method of developing a treatment for a chronic inflammatory disease in a patient.

### CROSS-REFERENCE TO RELATED APPLICATIONS

This application claims the benefit of (a) U.S. Provisional Application No. 63/376,400, filed Sep. 20, 2022 and (b) U.S. Provisional Application No. 63/500,901, filed May 8, 2023. Both applications are incorporated by reference herein in their entirety.

### BACKGROUND OF THE INVENTION

### (1) Field of the Invention

The present application generally relates to panels of gene and protein biomarkers. More specifically, gene and protein biomarker panels are provided that are useful for determining and developing treatments for chronic inflammatory diseases.

### (2) Description of the Related Art

Inflammatory reactions are well-regarded causes of bodily disorders and conditions and associated comorbidities, but early identification of the silent inflammatory reaction is elusive and not readily apparent. The present invention will provide very early indicators that a morbid disease process is occurring even before symptoms are apparent to the patient or the doctor. The invention will also allow confirmation of therapeutic response. The causative disease formula involving inflammatory genetic cytokines is common to the entire mammalian body, only the specific organ system is different—heart (myocarditis), brain (meningitis), liver (hepatitis), kidney (nephritis), placenta (vasculitis), etc.

### BRIEF SUMMARY OF THE INVENTION

Provided is a method of determining whether a patient has bladder cancer, colon cancer, or breast cancer. The method comprises
a) measuring an mRNA level of at least one gene in a tissue of the patient, wherein the at least one gene is KRT1, CCL27, SDK1, PPARG, IL1A, SMAD3, SLC6A3, NR3C1, KRT83, HFE2, CASP3, CACNA1C, NFKB2, CASP9, or any combination thereof,
b) comparing the mRNA levels determined in a) with an mRNA level of the at least one gene from a reference tissue from
   i) at least one subject with bladder cancer, and/or ii) at least one subject with colon cancer, and/or iii) at least one subject with breast cancer,
wherein A) the patient has bladder cancer if the mRNA level(s) determined in a) is similar to the mRNA level from b)i); the patient has colon cancer if the mRNA level(s) determined in a) is similar to the mRNA level from b)ii); and the patient has breast cancer if the mRNA level(s) determined in a) is similar to the mRNA level from b)iii).

Also provided is a method of determining whether a patient has a chronic inflammatory disease. The method comprises determining expression levels in the patient of one or more genes or protein biomarkers listed in Table 1 or measurement of 80 HdG levels, wherein
(a) if the expression levels indicate inflammation according to Table 1, the patient has a chronic inflammatory disease, or
(b) if the expression levels do not indicate inflammation according to Table 1, the patient does not have a chronic inflammatory disease.

Further provided is a method of evaluating patient response to a treatment for a chronic inflammatory disease, the method comprising
a) determining expression of at least one or more genes or protein biomarkers listed in Table 1 or measurement of 80 HdG levels in a population of subjects without the chronic inflammatory disease;
b) determining expression in the patient of the at least one or more genes or protein biomarkers listed in Table 1 prior to the treatment; and
c) determining expression of the at least one or more genes or protein biomarkers after the treatment,
wherein i) the patient is responding to the treatment if expression of the at least one or more genes or protein biomarkers approaches the expression in a) after treatment, and
ii) the patient is not responding to the treatment if expression of the at least one or more genes or protein biomarkers does not approach the expression in a) after treatment.

Additionally provided is a method of developing a treatment for a chronic inflammatory disease in a patient. The method comprises
(a) obtaining a potential treatment medication;
(b) determining expression levels in the patient of one or more genes or protein biomarkers listed in Table 1 or measurement of 80 HdG levels prior to administering the medication;
(c) administering the medication to the patient;
(d) determining expression levels in the patient of the one or more genes or protein biomarkers after administering the medication; and
(e) establishing the medication as a treatment for the disease if a change in expression levels between step (b) and step (d) indicates that the medication reduces or increases inflammation.

### DETAILED DESCRIPTION OF THE INVENTION

### Definitions

Acute respiratory disease syndrome (ARDS): Condition in which fluid builds up in the tiny, elastic air sacs (alveoli) in your lungs. The fluid keeps your lungs from filling with enough air, which means less oxygen reaches your bloodstream. This deprives your organs of the oxygen they need to function.
Adaptogen: In traditional Chinese medicine, ginseng is usually described as an “adaptogen,” a substance that can assist an organism in overcoming various types of stress, restore balance or homeostasis and increase nonspecific resistance.
Antibody: A blood protein produced in response to and counteracting a specific antigen. Antibodies combine chemically with substances which the body recognizes as alien, such as bacteria, viruses, and foreign substances in the blood.
Antigen: a toxin or other foreign substance which induces an immune response in the body, especially the production of antibodies.
Apoptosis: the death of cells which occurs as a normal and controlled part of an organism's growth or development.
Atherosclerosis: A disease of the arteries characterized by the deposition of plaques of fatty material on their inner walls.
Carcinogenesis: The initiation of cancer formation.
COPD: Chronic obstructive pulmonary disease.
CRP: C-reactive protein. A protein made by your liver. It is sent into your bloodstream in response to inflammation.
Cytokines: Small proteins that are involved in signals sent between cells.
Examples: nuclear factorE2 (Nrf2), superoxide dismutase (SOD), TNF-α and NF-κB, IFN-γ, and IL-6.
DNA: Deoxyribonucleic acid, a self-replicating material which is present in nearly all living organisms as the main constituent of chromosomes. It is the carrier of genetic information.
Endogenous: Growing or originating from within an organism.
Endothelial syndrome: Damage to the inner lining of the blood vessel that leads to atherosclerosis.
Glaucoma: a condition of increased pressure within the eyeball, causing gradual loss of sight.
Glutathione (GSH): an antioxidant in plants, animals, fungi, and some bacteria. Glutathione is capable of preventing damage to
important cellular components caused by reactive oxygen species such as free radicals, peroxides, lipid peroxides, and heavy metals.
Hyperlipidemia: An abnormally high concentration of fats or lipids in the blood.
Glutathione reductase: A ubiquitous enzyme, which catalyzes the reduction of oxidized glutathione (GSSG) to glutathione (GSH).
Immune cells: Cells that are part of the immune system and help the body fight infections and other diseases. Immune cells develop from stem cells in the bone marrow and become different types of white blood cells.
Inflammatory molecules: Inflammatory mediators that are part of the complex biological response of body tissues to harmful agents, such as bacteria, viruses, damaged cells or toxic irritants.
Innate immunity: A person's physical, chemical, and cellular defenses against invasion or disease.
Insulin: A hormone that helps blood sugar enter the cells in your body where it can be used for energy. Without insulin, blood sugar cannot get into cells and builds up in the bloodstream.
LPS induced cells: LPS can stimulate cells to become inflamed.
Metabolic syndrome: A cluster of biochemical and physiological abnormalities associated with the development of cardiovascular disease and type 2 diabetes.
Minerals: Substances that are formed naturally in the Earth. Minerals are usually solid, inorganic, have a crystal structure, and form naturally by geological processes.
mRNA: Messenger RNA. Transfers information from DNA to the cell machinery that makes proteins.
miRNA: Small, non-coding RNA molecules that regulate expression of a person's genes.
Natural killer (NK) cells: A cell that can react against and destroy another cell without prior sensitization to it. An NK cell attaches to a target cell, releases chemicals that breach its cell wall, and causes it to lyse (break up).
Necrosis: the death of most or all the cells in an organ or tissue due to disease, injury, or failure of the blood supply.
Neutrophils: A type of white blood cell that protects against infection.
Nrf2: A protein that controls how certain genes are expressed. These genes help protect the cell from damage caused by free radicals (unstable molecules made during normal cell metabolism).
Nuclear Factor Kappa B (NF-κB): A protein complex that controls transcription of DNA, cytokine production and cell survival.
Oxidative Stress: an imbalance between the production of free radicals and the ability of the body to counteract or detoxify their harmful effects through neutralization by antioxidants.
Oxygen stress: Also referred to as oxidative stress.
Oxygen radicals: A type of unstable molecule that contains oxygen and that easily reacts with other molecules in a cell. A buildup of oxygen radicals in cells may cause damage to DNA, RNA, and proteins, and may cause cell death. An oxygen radical is a free radical. Also called reactive oxygen species.
Proteins: Any of a class of nitrogenous organic compounds that consist of large molecules composed of one or more long chains of amino acids and are an essential part of all living organisms, especially as structural components of body tissues such as muscle, hair, collagen, etc., and as enzymes and antibodies.
Senescence: The condition or process of deterioration with age.
SLE: Systemic lupus erythematosus. An autoimmune disease in which the immune system attacks its own tissues, causing widespread inflammation and tissue damage in the affected organs. It can affect the joints, skin, brain, lungs, kidneys, and blood vessels.
SOD: Superoxide dismutase; A metal-containing antioxidant enzyme that reduces harmful free radicals of oxygen formed during normal metabolic cell processes to oxygen and hydrogen peroxide
Stem cells: Stem cells are special human cells that can develop into many different cell types, from muscle cells to brain cells. In some cases, they also can repair damaged tissues.
T1DM: Type 1 diabetes mellitus. A condition in which the pancreas does not make insulin or makes very little insulin.
T2DM: Type 2 diabetes mellitus. A condition in which the cells cannot use blood sugar efficiently to meet the body's needs.
Th 1: Cells that promote an immune response against viruses and bacteria.
Th 2: Cells that promote an immune response against parasites and also facilitate tissue repair.
TNF-α: Tumor Necrosis Factor alpha. An inflammatory cytokine produced by macrophages/monocytes during acute inflammation and is responsible for a diverse range of signaling events within cells, leading to necrosis or apoptosis. The protein is also important for resistance to infection and cancers. As a pro-inflammatory cytokine, TNF is secreted by inflammatory cells, which may be involved in inflammation-associated carcinogenesis.
Transcription factors: Proteins that help turn specific genes “on” or “off”
Venous thromboembolism (VTE): A condition in which a blood clot forms most often in the deep veins of the leg, groin or arm (known as deep vein thrombosis, DVT) and travels in the circulation, lodging in the lungs (known as pulmonary embolism, PE).

The present invention provides new tools to help with an accurate diagnosis, disease monitoring, and evaluating response to treatment, especially in complex inflammatory reactions and disorders without available effective treatments, such as heart attacks, sudden cardiac death, preeclampsia, cancer, diabetes, arthritis, autoimmune, colitis, pulmonary disease, etc.

Mounting evidence suggests that inflammation is a common underlying cause of major diseases, including but not limited to—heart disease, diabetes, cancer, stroke, Alzheimer's disease, viral pneumonia, etc. (Hunter, 2012). Inflammation is part of the body's immune response. Immune cells produce antibodies, cytokines and other inflammatory molecules. Recent science has uncovered new inflammatory molecules and the pathways through which they interact (Fukata, 2007). Doctors and medical scientists now realize that chronic inflammation is crucial to disease development and that new avenues of diagnosis, treatment and/or prevention are needed (Uttara, 2009).

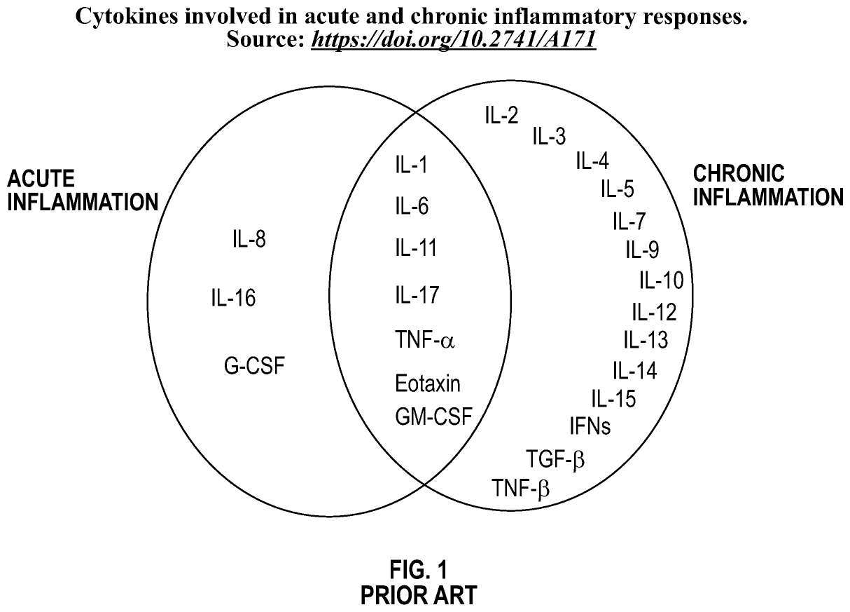

Inflammation is usually divided into two groups: acute inflammation and chronic inflammation. In many cases, these two groups are not well demarcated, and acute merges into chronic. Inflammation is mediated by various soluble factors, predominantly secreted polypeptides called cytokines. Some cytokines are specific to either acute or chronic inflammation while some are involved in both conditions (Feghali, 1997) (FIG. 1).

Why is chronic inflammation a silent killer, and possibly also acute inflammation? A traumatic injury, such as a sprain or broken bone, is obvious as a cause of an inflammatory reaction. The closing up of one's arteries is a silent inflammatory reaction. “We do not feel our arteries closing up until there is no blood supply and thus no oxygen to the heart muscle. We then experience a heart attack.” (Advanced Center, 2017).

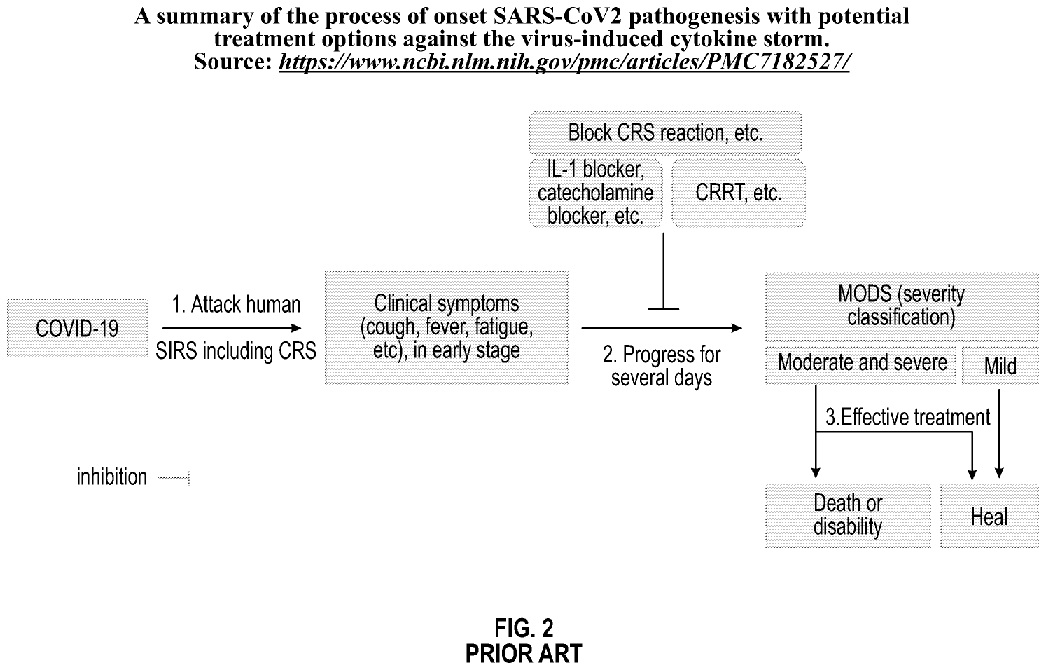

Inflammation is the response of tissues to trauma or injury and in the acute phase, there is increased blood flow and increased movement of fluid and cells out of blood vessels. Fluid, white blood cells, and inflammatory mediators, such as cytokines, accumulate in the involved area. A specific example is a condition called cytokine storm which occurs in the lungs of Covid infected patients. Cytokine storm occurs when one's immune system responds too aggressively to infection. “We propose a unifying definition for cytokine storm that is based on the following criteria: elevated circulating cytokine levels, acute systemic inflammatory symptoms, and secondary organ dysfunction beyond that which could be attributed to a normal response to a pathogen, if a pathogen is present” (Fajgenbaum, 2020). This acute inflammatory response in the Covid example is also known as “severe acute respiratory syndrome coronavirus 2 (SARS-CoV-2).” A conclusion in a review article succinctly summarizes the issues—“In summary, further experimentation is required to understand the changes in the immune response of patients with COVID-19 infection and the mechanisms of abnormal cytokine expression in COVID-19 pneumonia. Accurate prediction and targeted intervention during the course of COVID-19 pneumonia will be essential to improve patient survival (FIG. 2), (Sun, 2020).

Acute vs Chronic Inflammation
Source: https://www.labmed.org.tw/upfiles/issues/20091221155246.pdf

| | Acute Inflammation | Chronic Inflammation |
|---|---|---|
| Causes | Pathogens | Persistent acute |
| | Injured tissues | inflammation |
| | | Autoimmune disorders |
| | | Exposure toxins |
| Cells Involved | Monocytes | Monocytes |
| | Macrophages | Macrophages |
| | | Lymphocytes |
| | | Plasma cells |
| | | Fibroblasts |
| Mediators | Vasoactive amines | Cytokines |
| | Eicosanoids | Growth factors |
| | Neutrophils | Reactive oxygen species |
| | Prostaglandins | Hydrolytic enzymes |
| Major | Production of | Microalbuminuria |
| Inflammatory | proinflammatory | Oxidative stress |
| Events | cytokines | Lipid Peroxidation |
| Associated | IL-6 | IL-6 |
| Markers | CRP | CRP |
| | IL-8 | suPAR |
| | IL-12 | VCAM-1 |
| | TNFA- α | ICAM-1 |
| Onset | Immediate | Delayed |
| Duration | Minutes - Days | Months - Years |
| Outcome | Resolution | Tissue destruction |
| | Abscess formation | Fibrosis |
| | Chronic inflammation | |

Acute inflammation is a protective, immediate response to tissue damage that limits the damage caused by the noxious stimulus. Chronic inflammation refers to along-term, slow response caused by a combination of several noxious stimuli including infection and tissue destruction. The degree to which chronic inflammation contributes to a particular injury depends on the cause of the injury and how well the body can repair itself. Left unchecked, chronic inflammation can damage healthy cells and tissues, resulting in internal scarring and tissue death. Ultimately this can lead to potentially disabling or life-threatening illnesses.

Specific disease examples follow which present and explain a unifying definition of inflammatory causation of disease and the potential application of the gene and protein panels provided herewith to diagnose, monitor, and treat acute and chronic inflammatory diseases/conditions.

### Preeclampsia:

Preeclampsia is a pregnancy-specific, hypertensive disorder that is associated with high maternal morbidity and mortality. It is characterized by hypertension, proteinuria, and endothelial dysfunction, and it occurs in approximately 5% of all pregnancies and up to 10% of the cases of stillbirths worldwide. Preeclampsia is often associated with an increased risk of HELLP syndrome (Hemolysis, Elevated Liver enzymes, and Low Platelets), which can lead to serious complications for both mother and baby. The exact etiology of preeclampsia is unknown, but there are several theories that attempt to explain the pathogenesis of this syndrome. One of these theories involves chronic immune activation leading to increased production of inflammatory cytokines by pro-inflammatory T cells, and a decrease in regulatory and anti-inflammatory cytokines, which further promotes an inflammatory state during preeclampsia (Harmon, 2016). Treatment includes low-dose aspirin, calcium channel blockers, and anti-hypertensive medications. There is no standard treatment for preeclampsia because there are so many different causes. The only effective way to prevent pregnancy-related hypertension and preeclampsia is to avoid pregnancy.

The exact cause of preeclampsia is unknown. Some sources report that the condition may develop when there is an issue with the placenta (NHS, 2021). It is believed that an imbalance in certain hormones signals blood vessels supplying the placenta to respond differently than normal, which causes a narrowing of blood flow (Felman, 2021). It could also possibly be caused by problems with placenta development because some blood vessels are abnormally small and have abnormal connections from the baby's acupressure points. Although further research would need to be conducted, it has been concluded that treatment for preeclampsia focuses on ensuring maternal and fetal well-being.

Women who are at risk for developing pre-eclampsia include those who have diabetes, high blood pressure or kidney disease before pregnancy, have an autoimmune condition, such as lupus or antiphospholipid syndrome, or have high blood pressure or pre-eclampsia in a previous pregnancy (NHS, 2021).

Furthermore, preeclampsia is a multi-factorial disease that has been associated with chronic immune activation. Increased levels of pro-inflammatory cytokines have been found in plasma and tissues in preeclamptic women. These inflammatory changes have been linked with endothelial dysfunction and vascular reactivity, as well as other symptoms of preeclampsia such as edema, hypertension, and liver dysfunction (Harmon, 2016).

The link between inflammation and preeclampsia is important because inflammatory conditions are common in chronic diseases like diabetes, heart disease, and atherosclerosis (hardening of the arteries). The placenta releases a hormone called placental lactogen (PL) that causes the mother's blood pressure to drop. This drop in blood pressure allows more blood to flow to the uterus and placenta, which can result in an increase in the mother's inflammatory response.

When the immune system encounters an illness or injury, it activates inflammatory cells and cytokines (substances that stimulate more inflammatory cells) to help the body trap bacteria and other offending agents or start healing injured tissue.

The inflammation involved with preeclampsia may be due to a variety of causes, including
Stress hormones, such as cortisol and adrenaline (epinephrine) from the body's response to stress
The immune system reacting to bacteria in the body
Abnormal fluid retention caused by obesity (pregnancy weight gain) or diabetes (high blood sugar levels)

However, the exact mechanisms that cause this inflammation are not known. It is believed that this type of inflammation may be related to increased production of an immune system protein called tumor necrosis factor-alpha (TNF-α) in response to inflammation. TNF-α may also cause changes in gene expression (i.e., how genes are turned on or off) that lead to an overproduction of cytokines.

The placenta is the maternal-fetal interface during pregnancy. Placental dysfunction is a major contributor to adverse pregnancy outcomes, including preeclampsia, fetal growth restriction, fetal demise, and preterm birth. Placental ischemia refers to a lack of blood flow to the placenta, which compromises the delivery of oxygen and nutrients to the fetus. When placental ischemia occurs, the cells of placental villi are deprived of oxygen and nutrients, resulting in cell death. This phenomenon has been observed in pre-eclampsia gravidarum, a syndrome that is characterized by maternal hypertension and edema as well as fetal organ dysfunction. “The resultant placental ischemic microenvironment is inherently linked to increased production and secretion of deleterious soluble mediators that provoke extensive maternal inflammation and endothelial dysfunction,” (Williamson, 2019).

When a placenta is injured, it secretes proinflammatory mediators to the maternal circulation. This can result in a pro-inflammatory and antiangiogenic environment that contributes to endothelial dysfunction, thereby increasing susceptibility to preeclampsia. “This imbalance between pro-inflammatory and regulatory cytokines is associated with the placental ischemia that occurs during a preeclamptic pregnancy. This imbalance worsens as the pregnancy progresses,” (Harmon, 2016). We know that the placenta is essential to the development of the fetus, supplying it with oxygen and nutrients and disposing of waste. However, we also know that the placenta is associated with maternal cardiovascular disease and hypertension, which can lead to preeclampsia and other pregnancy complications. The reason for this association has never been clear.

Ultimately, maternal pre-eclampsia and the development of placental insufficiency are associated with oxidative stress and mitochondrial dysfunction. These findings may help explain why pre-eclampsia has such deleterious effects on both mother and fetus through mechanisms involving oxidative stress and mitochondrial dysfunction.

Because of their distinct biochemical and physical properties, exosomes have been shown to be a novel class of vesicles that can act in intercellular communication. Exosomes are extracellular vesicles that cells generate to serve as mediators of near and long-distance intercellular communication in health and disease and affect various aspects of cell biology. Exosomes carry nucleic acids, proteins, lipids, and metabolites. Those that come from healthy cells help protect them against infection. However, those that come from diseased cells can cause diseases such as cancer. Validated studies have discovered that placental-derived exosomes are present in the maternal circulation and their concentration increases throughout normal pregnancy. The total number of exosomes present in maternal plasma increases in women between 11 and 14 weeks gestation who were subsequently identified as having gestational diabetes mellitus. The researchers suggest that changes in the exosome concentration present in maternal circulation at the early stages of pregnancy are important in the development of pregnancy complications such as preeclampsia later in gestation.

A recent study revealed an increased risk of pregnancy complications including preterm birth, cesarean delivery, and preeclampsia associated with Covid-19 infection during pregnancy (NIH, 2022). Coronavirus disease, also known as Severe Acute Respiratory Syndrome (SARS), is an infectious disease caused by the SARS-CoV-2 virus. Pneumonitis, or inflammation of the lungs, can result from an irritant such as airborne mold or a chemotherapy drug. This inflammation makes it difficult for oxygen to pass through tiny air sacs (alveoli) in the lungs into the bloodstream.

Pregnant women with COVID-19 had a 76% greater chance of pregnancy-induced high blood pressure, known as preeclampsia or eclampsia (Kaiser, 2021). This condition can be life-threatening for both mother and baby. The study also found that women who had antibodies to the virus were nearly six times more likely to develop high blood pressure within the first three months of their pregnancy.

Recent disturbing scientific study results at Thomas Jefferson University have revealed that adverse events after Covid-19 vaccination may be due to lipid nanoparticles (LNPs) that are used in the vaccine to transport the mRNA in the vaccine (Zhang, 2022; Igyarto, 2021). “It was originally intended that the LNPs discreetly deliver mRNA sequences into the cells to produce spike proteins, and in doing so, form immunity against the COVID-19 virus. However, many studies in mice have since found that the LNPs, claimed to be non-toxic and safe, are actually highly inflammatory.” Nevertheless, growing data of reported adverse health effects in humans following COVID-19 vaccination warrant further research. Examination of the overlaps between health implications for mice and humans is also needed.

“Considering the broad exposure of a large proportion of human populations to vaccines based on this novel (mRNA) technology, more studies are warranted to fully understand its overall immunological and physiological effects. Determining this platform's short and long-term impact on human health would help optimize it to decrease its potentially harmful effects,” the authors concluded.

Ergothioneine, a sulfur-containing amino acid, has garnered attention as a possible therapeutic agent. The potential of such an approach is based on the preferential accumulation of ergothioneine within tissues undergoing significant oxidative stress, coupled with its proven ability to protect against oxidative stress and associated tissue damage in animal studies. This is partly because of its preferential accumulation within tissues undergoing significant oxidative stress and also due to the fact that it has been touted for multiple health benefits, ranging from protection against environmental toxicity to anti-aging. Although extremely promising in animal models, the evidence for ergothioneine's efficacy in humans is still quite limited.

Ergothioneine, also known as thiostrepton, is a naturally occurring amino acid and is a thiourea derivative of histidine. It is synthesized by the enzyme ergothioneine synthase. The sulfur atom on the imidazole ring of ergothioneine is thought to be essential to its biological activity. The structure of this compound was first identified in 1934. This compound occurs in relatively few organisms, notably Actinomycetota, “Cyanobacteria”, and certain fungi. It functions as an antioxidant in the cell membranes of these organisms, protecting them from free radicals.

The placenta plays a critical role in the transport of oxygen, carbon dioxide, and nutrients across the maternal-fetal interface. As stated previously, placental ischemia occurs when there is insufficient blood flow to the placenta, causing it to cease functioning. The resulting hypoxia can cause oxidative stress in the mother and fetus by inhibiting protein synthesis and disrupting cell membranes. This may lead to increased production of reactive oxygen species (ROS) and free radicals, which damage proteins and lipid components. Recent evidence suggests that mitochondrial dysfunction may be a prime mediator of this process. Mitochondria are located within every cell in the body and play a key role in maintaining cellular homeostasis by providing energy for most cellular processes. In addition, mitochondria generate ROS as part of their normal metabolism. These ROS can cause damage to lipids, proteins, and DNA if they accumulate too much or persist for too long.

The 2019 ergothioneine study by Williamson et al, provided evidence of the therapeutic potential of ergothioneine in a preclinical model of preeclampsia. “Ergothioneine improved both hypertension and fetal weight in the RUPP rat model of preeclampsia. Furthermore, ergothioneine treatment significantly altered mitochondrial function in both the kidney and placental tissue, which may, in part, be responsible for the beneficial effects on phenotypic features of preeclampsia in the RUPP model. Given its favorable safety profile, its long half-life and resistance to auto-oxidation and multiple mechanisms of action, not least its ability to regulate mitochondrial function, further studies are needed to explicitly define the protective mechanisms of ergothioneine in treating preeclampsia in humans,” (Williamson, 2019).

Additional evidence of the association of ergothioneine with preeclampsia is the finding of increased levels of ergothioneine in the red blood cells of preeclamptic women (Turner, 2009). A rat study using the RUPP procedure showed that the addition of ergothioneine to the water of the rat dampened the inflammatory response in preeclampsia. This study highlighted the potential of L-Ergothioneine as a possible treatment for preeclampsia (Morillon, 2020).

In summary, L-Ergothioneine is found in food, especially mushrooms. The absence of toxicity, high potency, and broad spectrum antioxidant properties, together with high water solubility and stability at physiologic pH, make ergothioneine a very attractive candidate for protecting endothelial cells against oxidative stress (Li, 2014). This compound has the capability for the treatment and/or prevention of oxidative stress-associated cardiovascular diseases and perhaps even preeclampsia.

### Bladder Cancer

It is well accepted that inflammatory reactions are associated with the overproduction of free radicals, such as cytokines, as well as the loss of the antioxidant system's efficiency in protecting the body. Free radicals can act as a molecular trigger of the mechanism of inflammation after the initial insult. Oxidative stress mediates the activation of NFkB inducing in turn the transcription of certain genes promoting cytokine production. The release of these cytokines results in the enhancement of the inflammatory response (Closa, 2004).

Bladder cancer (BC) is one of the most diagnosed cancers among people aged over 65 years. BC is the fourth most common type of cancer in men and the eighth-most commonly diagnosed malignancy in women worldwide. Information about what causes BC is still under investigation, but recent scientific reports indicate that oxidative stress and reactive oxygen species are involved in the formation, development, and progression of this cancer (Phaniendra, 2015). It has been demonstrated that reactive oxygen/nitrogen species (ROS/RNS) have been involved in the development of various kinds of cancer due to irreversible damage to cellular and extracellular macromolecules, including DNA and associated mutations (Islam, 2019). A clinical study at the University of Lodz in Poland analyzed the plasma levels of protein carbonyls, thiol groups, 3-nitrotyrosine, lipid peroxidation, as well as non-enzymatic plasma antioxidant capacity using DPPH and ABTS+radicals. The study confirmed that all analyzed biomarkers are higher in enrolled BC patients than in healthy subjects. “Furthermore, our findings demonstrate a positive correlation between the degree of bladder cancer progression and the level of oxidative stress.” (Wigner, 2021). BC patients in another study showed increased concentrations of markers characterizing oxidative stress and the inflammatory response, such as MDA, AOPP, and CRP as well as markers of angiogenesis, such as ANG, combined with the decreased activity of endogenous antioxidants (Sawicka, 2020) The majority of the newly diagnosed cases of BC will be diagnosed prior to muscle invasion, thus potentially completely curable. Unfortunately, >20% of patients initially diagnosed with non-muscle-invasive bladder cancer will eventually die of their disease despite local endoscopic surgery (Utz, 1980). Mycobacterium Bovis bacillus Calmette-Guerin (BCG) has been used for the treatment of bladder cancer since 1976 and continues to be at the forefront of therapeutic options for this malignancy. Despite its success and worldwide acceptance, the antitumor effector mechanisms remain elusive. (Simons, 2008). The measurement of cytokine expression as suggested in this patent may be the answer to determining patient response to BCG therapy. Findings from the Department of Urology, University of Iowa demonstrated that TNF-related apoptosis-inducing ligand (TRAIL) is induced by BCG treatment, and TRAIL was expressed on polymorphonuclear neutrophils (PMN) in the urine obtained from patients after intravesical BCG instillation (Ludwig, 2004).

### Cardiovascular Conditions and Diseases

Two chronic disease conditions which severely impact society in terms of morbidity, mortality and economic impact are cardiovascular disease (CVD), and endothelial syndrome or atherosclerosis. Evidence has accumulated about the association between inflammation, oxidative stress and proinflammatory cytokines in these disorders. While atherosclerosis was originally considered a disease caused by impaired lipid storage, it has since been convincingly demonstrated that it is a result of misguided inflammatory processes. “Current evidence supports a central role for inflammation in all phases of the atherosclerotic process” (Libby, 2002). The control of the risk factors is the main cost-effective available measure for preventing major events associated to CVD (Kyu, 2018).

Atherosclerosis, a chronic vascular disease, provides a clear picture of association with inflammation, oxidative stress, and dysfunction of the endothelial blood vessel lining (endothelial dysfunction). The event that initiates plaque formation is the accumulation of modified LDL in the intima and atherosclerosis is the result of the immune and inflammatory responses to this phenomenon. Inflammatory cells and cytokines play a key role in atherosclerotic plaque initiation and progression. However, “The complicated mechanisms associated with inflammatory responses related to atherosclerosis remain largely unclear. (Wu, 2017).

It has been shown that several pro-inflammatory cytokines, such as IL-6, IL-18, and TNF-α are each associated with ˜10-25% higher risk of non-fatal myocardial infarction (MI) or coronary heart disease (CHD) death. These risk factors are independent of conventional risk factors and “in an approximately log-linear manner.” (Kaptoge, 2014).

A study analyzing the human arterial tissue proteomics identified several vascular and plasma biomarkers related to early atherosclerosis including TNF-α, insulin receptor, PPARα, and PPARγ protein networks, predictors of both development and site of atherosclerosis and CVD (Herrington, 2016).

Intercellular communication is an essential hallmark of chronic disease and it is important to create an integrative computational tool that will identify unmet personalized human needs related to development of chronic diseases. mRNAs and miRNAs may be the answer in their ability to regulate gene expression post transcriptionally and function within the cells in which they are transcribed. Alexander and colleagues showed findings that provide strong evidence that endogenous microRNAs undergo a functional transfer between immune cells and constitute a mechanism of regulating the inflammatory response (Alexander, 2015).

A key element to the diagnostic puzzle and potential ability to measure genetic susceptibility to chronic disease is translational bioinformatics (TBI). According to the American Medical Informatics Association (AMIA), translational bioinformatics (hereafter “TBI”) is “the development of storage, analytic, and interpretive methods to optimize the transformation of increasingly voluminous biomedical data, and genomic data, into proactive, predictive, preventive, and participatory health” (http://www.amia.org/applications-informatics/translational-bioinformatics). Translational bioinformatics methods continue to make an actual difference in patients' lives. The infrastructure, information technology, policy, and culture need to catch up with some of the technological advances (Tenenbaum, 2016).

### Vasculitis

Vasculitis is a condition that involves inflammation of the blood vessels. A variety of conditions can cause vasculitis, including infections, autoimmune diseases, and even certain types of cancer. Whatever the underlying cause of the inflammation, its ultimate effect is to narrow the lumen—that's the space inside a blood vessel that is available for blood flow—which in turn restricts circulation.

There are several different kinds of vasculitis: necrotizing (or “leaky”) arteritis, coronary artery vasculitis, eosinophilic granulomatosis with polyangiitis (EGPA), and granulomatosis with polyangiitis (GPA). Most cases of vasculitis are caused by an abnormal immune response against some kind of irritant in the body—it could be an infection, or it could be another substance entirely. In necrotizing arteritis, for example, the body thinks that an infection has entered through the blood vessels and mounts an attack against it; in this case, however, it happens to target a type of cell called smooth muscle cells that line the lumen walls. The result is leakage of fluids from between the cells and into surrounding tissue, making it swell up and potentially become inflamed.

Systemic vasculitides are a heterogeneous group of diseases that affect the blood vessels with lesions that affect the entire body. Examples of systemic vasculitides include giant cell arteritis, Takayasu's arteritis, and polyarteritis nodosa. These conditions are not curable and the therapeutic approaches are mainly symptomatic. The symptoms of systemic vasculitides include fever, weight loss, fatigue, night sweats, and constitutional symptoms such as malaise, widespread pain with tenderness to palpation, and headaches.

Moreover, systemic vasculitides show a complex etiology in which both environmental and genetic factors play a major role in their development. A number of viruses have been implicated in their pathogenesis; however, many systemic vasculitides appear to arise in the absence of infection. These conditions could be considered to be autoimmune diseases because they are thought to have a strong genetic component; however, other factors such as environmental triggers play important roles in their development.

This distinct family of disorders is associated with an increased risk of developing pulmonary arterial hypertension and superimposed infection in patients with SLE. In addition, the presence of the HLA-B27 phenotype is strongly associated with the development of these conditions. However, despite their increased prevalence in patients with SLE, it was surprising to note that some systemic vasculitides may be triggered by certain drugs such as NSAIDs, and others can occasionally present as SLE-like disease process in which features of both conditions are present at the same time.

Biomarkers are indicators of specific biological activity and thereby provide an opportunity to establish a characteristic biochemical profile of disease pathogenesis, progression, and/or treatment. Analysis of individual mRNA inflammatory genetic markers as well as novel patterns of mRNA inflammatory genetic markers provide an opportunity to establish a characteristic biochemical snapshot or profile of disease pathogenesis, progression, and/or treatment.

Table 1 below highlights specific mRNA cytokines, and metabolic protein signals associated with conditions including but not limited to—heart disease, diabetes, autoimmune disorders, preeclampsia, bladder cancer, colon cancer, and breast cancer.

TABLE 1
Biomarkers and Potential Treatment Options for Common Chronic Inflammatory Diseases

| | Preeclampsia | Bladder Cancer | Breast Cancer | Colon Cancer | Cardiac Diseases |
|---|---|---|---|---|---|
| Prediction | Up Regulated (↑) | | | | |
| of Protein | ABCA1, ALP, | 80HdG, IL-10, | CRP, G-CSF, | 8OHdG, Bcl-2, | 8OHdG, |
| Signals | AT1-AA, B, | IL-17RB, IL- | IL-1, IL-13, | Bcl-XL, EGFR, | ABCA1, |
| During | Calretinin, | 17RE, IL-1F5, | IL-17, IL-18, | HFE, IL-1, | ADAMTS4, |
| Inflammation | CASP-1, CD55, | IL-1RAPL1, | IL-19, IL-19, | IL-18, IL-6, | CASP-1, |
| | Ebi3, EGFR, | IL-20, IL- | IL-2, IL-20, | IL-8, MUC1, | CRP, DMT1, |
| | Endoglin, EPO, | 22RA1, IL-26, | IL-21, IL-23, | NFKB1, p21, | EPO, Fas, |
| | ET-1, Factor, | IL-28AR1, IL- | IL-32, IL-4, | p27, PDL1, | GCKR, GF1, |
| | Flt-1, HSP90, | 4, IL-5, IL-6, | IL-6, IL-7, | SIRT1, STK11, | HAMP, IFN-γ, |
| | HtrA, IL-1, IL- | IP10, PDGF, | NFKB1, PDL1, | Survivin, | IL-1, IL-19, |
| | 18, IL-6, IL-8, | PDL1, SIRT1, | SKA2, TGF-α, | TGFB1, TNF, | NFKB2, PDL1, |
| | Nox4, p22- | SKA2, TRAF- | TGF-β, TGFB1, | TNF-α, VEGF, | PPARG, RGS2, |
| | phox, PAI1, | 2, TRAIL, | TNF-α | XIAP | SIRT1, |
| | PAX8, PBEF, | VEGF | | | SLC40A1, |
| | PPARG, PSM, | | | | ST-2, TGFB1, |
| | Ptx3, RGS2, | | | | TNF, TNF-α, |
| | ROS, sEng, | | | | TRAIL, VEGFA |
| | SKA2, TFPI2, | | | | |
| | TNF, TNF-α, | | | | |
| | Vimentin, | | | | |
| | WT1, XIII | | | | |
| | Down Regulated (↓) | | | | |
| | 14-3-3β, | B2M, BCL2, | 8OHdG, Ca2+, | CASP-1, | Ca2+, GH1, |
| | ADAMTS4, | CASP-1, | CASP-1, CASP-3, | CASP-10, | HFE2, IL-10, |
| | AngII, | CASP-8, | CASP-8, IL-10, | CASP-8, | IL-17, IL-18, |
| | AnnexinXI, | CCL14, CD8A, | IL-11, IL-1β, | CASP-9, GH1, | IL-1β, IL-6, |
| | Bcl-6, Bcl-xL, | CFD, CFH, | IL-22, IL-8, | KLF4, PTEN, | IL-8, L-type, |
| | CASP-8, | CFI, | L-type, TGF-β, | TRAIL | PAX8, TGF-β |
| | cdc25B, CREB, | CLEC10A, | TNF | | |
| | Glypican3, | CXCL12, | | | |
| | ICAM-1, IFN- | FCER1A, | | | |
| | γ, IGFBP3, IL- | HLA-DPA1, | | | |
| | 1β, MetRS, | HLA-DPB1, | | | |
| | NFkBp65, | HLA-DQA1, | | | |
| | Notch4, p- | HLA-DRA, | | | |
| | MAPK(Erk1/2), | HLA-DRB4, | | | |
| | p-PKCα/βII, | IL-1, IL-10RA, | | | |
| | PAPP-A, | IL-11RA, IL-15, | | | |
| | PCNA, PDEF, | IL-16, IL-18R1, | | | |
| | PIGF, sFLT1, | IL-18RAP, IL- | | | |
| | SIRT1, TRAIL | 1RA, IL-6ST, | | | |
| | | IL-7R, KRT83, | | | |
| | | NFKB1, NFKB2, | | | |
| | | TLR3, TLR7, | | | |
| | | TNF | | | |
| Potential | Panel Results Indicate Inflammation | | | | |
| Treatments | Boron Citrate | Mitomycin C | Everolimus | Cetuximab | Arcalyst |
| | Hydralazine | Quercetin | Methotrexate | Fluorouracil | Atenolol |
| | Labetalol | dihydrate | Palbociclid | Ipilimumab | Coenzyme |
| | Magnesium | Resveratrol | Quercetin | Oxaliplatin | Q10 |
| | Sulfate | Tecentriq | Dihydrate | Pembrolizumab | (CoQ10) |
| | Zinc Picolinate | | Resveratrol | Quercetin | Resveratrol |
| | | | | Dihydrate | Vyndaqel |
| | | | | Resveratrol | |
| | Panel Results Do Not Indicate Inflammation | | | | |
| | Aspirin-81 | Balversa | Evista | Glutathione | Glutathione |
| | Folic Acid (L- | Glutathione | Glutathione | (GSH) | (GSH) |
| | Methylfolate) | (GSH) | (GSH) | Selenium (SE) | Jardiance |
| | Vitamin D | Pembrolizumab | Letrozole | Vitamin A | Leqvio |
| | | Selenium (SE) | Raloxifene | Vitamin C | Thiamin |
| | | Vitamin A | Hydrochloride | Vitamin D | Vascepa |
| | | Vitamin C | Selenium (SE) | | |
| | | Vitamin D | Vitamin A | | |
| | | | Vitamin C | | |
| | | | Vitamin D | | |

Another biomarker that indicates inflammation is 80 HdG, or 8-hydroxy-2′-deoxyguanosine. 80 HdG is a chemically modified nucleotide that is formed when DNA is damaged by oxygen radicals. The presence of 80 HdG in DNA indicates oxidative damage to cellular components. In other words, it's a measure of oxidative stress in the body.

Determination of the level of any one or combination of the above mRNAs or protein biomarkers provide insight into the diagnosis or monitoring of progression of the subject diseases or disorders.

Thus, in some embodiments, a method of determining whether a patient has bladder cancer, colon cancer, or breast cancer is provided. The method comprises
a) measuring an mRNA level of at least one gene in a tissue of the patient, wherein the at least one gene is KRT1, CCL27, SDK1, PPARG, ILIA, SMAD3, SLC6A3, NR3C1, KRT83, HFE2, CASP3, CACNA1C, NFKB2, CASP9, or any combination thereof;
b) comparing the mRNA levels determined in a) with an mRNA level of the at least one gene from a reference tissue from
   i) at least one subject with bladder cancer, and/or ii) at least one subject with colon cancer, and/or iii) at least one subject with breast cancer,
wherein A) the patient has bladder cancer if the mRNA level(s) determined in a) is similar to the mRNA level from b)i); the patient has colon cancer if the mRNA level(s) determined in a) is similar to the mRNA level from b)ii); and the patient has breast cancer if the mRNA level(s) determined in a) is similar to the mRNA level from b)iii). See Example 3 below.

These determinations can be made by any means known in the art. In some embodiments, mRNA levels are determined by a next generation sequencing method, e.g., pyrosequencing, sequencing by reversible terminator, sequencing-by-ligation mediated by ligase enzymes, or real-time sequencing using phospholinked fluorescent nucleotides, etc. See, e.g., www.sciencedirect.com/topics/medicine-and-dentistry/next-generation-sequencing.

In some embodiments, the at least one gene is KRT1, CCL27, SDK1, PPARG or any combination thereof, and the method determines whether the patient has breast cancer or colon cancer. In some of these embodiments, the at least one gene is KRT1, CCL27 or the combination thereof.

In other embodiments, the at least one gene is IL1A, SMAD3 or the combination thereof, and the method determines whether the patient has bladder cancer or colon cancer.

In additional embodiments, the at least one gene is IL1A, CCL27 or the combination thereof, and the method determines whether the patient has bladder cancer or breast cancer.

In further embodiments, the at least one gene is SLC6A3, NR3C1, KRT1, KRT83, HFE2, CCL27, IL1A, SMAD3, CASP3, SDK1, CACNA1C, NFKB2, PPARG, CASP9, or any combination thereof, and the method determines whether the patient has bladder cancer, breast cancer or colon cancer. In some of these embodiments, the at least one gene is SLC6A3, NR3C1, KRT1, KRT83, HFE2, CCL27, or any combination thereof.

Any appropriate tissue can be utilized to measure the mRNA levels in these embodiments.

In some of these embodiments, the tissue is a saliva, blood, cell or urine sample. In other embodiments, the tissue is a sample of the cancer, e.g., from the primary tumor or from a metastatic tumor.

In some embodiments, the method further comprises recommending a treatment for bladder cancer if the mRNA level(s) determined in a) is similar to the mRNA level from b)i); recommending a treatment for colon cancer if the mRNA level(s) determined in a) is similar to the mRNA level from b)ii); or recommending a treatment for breast cancer if the mRNA level(s) determined in a) is similar to the mRNA level from b)iii). See, e.g., Table 1 and below for nonlimiting examples of treatments that can be recommended.

In other embodiments, the method further comprises treating the patient for bladder cancer if the mRNA level(s) determined in a) is similar to the mRNA level from b)i); treating the patient for colon cancer if the mRNA level(s) determined in a) is similar to the mRNA level from b)ii); or treating the patient for breast cancer if the mRNA level(s) determined in a) is similar to the mRNA level from b)iii). See, e.g., Table 1 and below.

### Current Regulatory Landscape for Anticancer Treatment:

Treatment plans for most cancers may include a combination of chemotherapy, targeted cancer cell therapy, immunotherapy, surgery, and radiation therapy, which can be used to slow the spread of the disease and shrink a cancerous tumor. Palliative care will also be important to help relieve symptoms of cancer and side effects of treatment.

Chemotherapy uses strong medicines to kill cancer cells. For example, chemotherapy for colon cancer is usually given after surgery if the cancer is large or has spread to the lymph nodes.

Chemotherapy can kill cancer cells that might be left after surgery. This helps reduce the risk of the cancer coming back. Chemotherapy might also be used before surgery to shrink a large cancer so that it's easier to remove. Chemotherapy also can be used to relieve symptoms of colon cancer that can't be removed with surgery or that has spread to other areas of the body. Sometimes it's used with radiation therapy. Chemotherapy usually works by attacking rapidly dividing cells. This means that chemotherapy can harm not only cancer cells but also healthy cells that are dividing rapidly, like the ones that cause your hair to grow.

Radiation therapy uses powerful energy beams to kill cancer cells. The energy can come from X-rays, protons or other sources. Radiation therapy can shrink a large cancer before an operation to make it easier to remove. When surgery isn't an option, radiation therapy might be used to relieve symptoms, such as pain. Some people have radiation and chemotherapy at the same time.

Targeted therapy uses medicines that attack certain chemicals in cancer cells. By blocking these chemicals, targeted treatments can cause cancer cells to die.

Immunotherapy is a treatment with medicine that helps the body's immune system kill cancer cells. Cancer cells survive by hiding from the immune system. Immunotherapy helps the immune system cells find and kill the cancer cells.

### Colon Cancer:

Surgery: The treatment of choice for colon cancer is surgical resection, which involves removing the cancer through surgery. Surgery is performed to remove the cancer completely and reconstruct the bowel, if possible, so a patient's postoperative bowel function is normal or near normal.

Standard chemotherapy for colorectal cancer: Capecitabine (Xeloda), a pill that is changed into 5-FU once it gets to the tumor. Irinotecan (Camptosar) Oxaliplatin (Eloxatin) Trifluridine and tipiracil (Lonsurf), a combination drug in pill form.

Immunotherapy is usually reserved for advanced colon cancer.

### Breast Cancer:

Breast cancer is primarily treated with surgery and often combined with chemotherapy, radiation therapy or both. It may also include other treatment options like targeted therapy, proton therapy and angiogenesis inhibitors. Surgery is often the first treatment for breast cancer.

Chemotherapeutic drugs include, but are not limited to, the following commonly used agents:
anthracyclines, such as doxorubicin
cyclophosphamide (Cytoxan®)
epirubicin (Ellence®)
fluorouracil
methotrexate (Rheumatrex®, Trexall®)
taxanes, such as paclitaxel (Taxol®)

Early-Stage Breast Cancer can be treated with combination chemotherapy. Combination drug therapy means that you receive more than one type of drug at a time. If a tumor becomes resistant to one drug, your treatment may still be effective because the tumor responds to the second or third drug in the combination you receive.

Combination therapy can be given before or after breast surgery. Most patients receive a combination of two or three drugs at the same time. Some of these drugs are breast cancer targeted therapies. These drugs work by targeting specific molecules involved in breast cancer development.

Drug combinations that medical oncologists commonly prescribe:

### Dose-Dense AC-T Chemotherapy

Doxorubicin and cyclophosphamide, followed by paclitaxel
Used to treat early-stage breast cancer, particularly in younger women or women with aggressive disease
Given intravenously before or after surgery

### Dose-Dense AC-TH Chemotherapy and Targeted Therapy

Doxorubicin and cyclophosphamide, followed by paclitaxel and trastuzumab (Herceptin®)
Used to treat early-stage HER2-positive breast cancer
Given intravenously before or after surgery

### Dose-Dense AC-THP Chemotherapy and Targeted Therapy

Doxorubicin and cyclophosphamide, followed by paclitaxel, trastuzumab, and pertuzumab (Perjeta®)
Used to treat early-stage breast cancer
Given intravenously before or after surgery

Chemotherapy drugs that are commonly prescribed for advanced breast cancer include:
albumin-bound paclitaxel (Abraxane®)
carboplatin
capecitabine (Xeloda®)
docetaxel
eribulin (Halaven®)
etoposide (VP16, Etopophos®)
gemcitabine (Gemzar®)
irinotecan (Camptosar®)
liposomal doxorubicin (Lipodox®)
paclitaxel
vinorelbine (Navelbine®)

### Bladder Cancer:

### Drugs Approved for Bladder Cancer

Adstiladrin (Nadofaragene Firadenovec-vncg)
Avelumab
Balversa (Erdafitinib)
Bavencio (Avelumab)
Cisplatin
Doxorubicin Hydrochloride
Enfortumab Vedotin-ejfv
Erdafitinib
Jelmyto (Mitomycin)
Keytruda (Pembrolizumab)
Mitomycin
Nadofaragene Firadenovec-vncg
Nivolumab
Opdivo (Nivolumab)
Padcev (Enfortumab Vedotin-ejfv)
Pemazyre (Pemigatinib)
Pembrolizumab
Pemigatinib
Sacituzumab Govitecan-hziy
Tepadina (Thiotepa)
Thiotepa
Trodelvy (Sacituzumab Govitecan-hziy)
Valrubicin
Valstar (Valrubicin)

### Drug Combinations Used in Bladder Cancer

Gemcitabine-Cisplatin
MVAC

Chemotherapy into the bladder (intravesical chemotherapy) is a treatment for non-muscle invasive bladder cancer. The medication is given through a flexible tube called a catheter, which goes into the bladder. Intravesical chemotherapy reduces the chance of the cancer coming back or spreading. Intravesical chemotherapy is different to chemotherapy treatment for muscle invasive bladder cancer. Muscle invasive means the cancer has spread into or through the muscle layer of the bladder.

Non muscle invasive bladder cancer or superficial bladder cancer means the cancer cells are only in the bladder's inner lining.

The most common treatments for non-muscle invasive bladder cancer are:
surgery
chemotherapy into the bladder
Instillation of a vaccine called BCG into your bladder

The most common regimens for systemic, or whole-body, chemotherapy to treat bladder cancer include cisplatin and gemcitabine, carboplatin (available as a generic drug) and gemcitabine, and MVAC, which combines 4 drugs: methotrexate (Rheumatrex, Trexall), vinblastine (Velban), doxorubicin, and cisplatin.

Also provided herewith is a method of determining whether a patient has a chronic inflammatory disease. The method comprises determining expression levels in the patient of one or more genes or protein biomarkers listed in Table 1 or measurement of 80 HdG levels, wherein
(a) if the expression levels indicate inflammation according to Table 1 or measurement of 80 HdG levels, the patient has a chronic inflammatory disease, or
(b) if the expression levels do not indicate inflammation according to Table 1 or measurement of 80 HdG levels, the patient does not have a chronic inflammatory disease.

In some embodiments,
(a) if the expression levels indicate inflammation according to Table 1 or measurement of 80 HdG levels, recommending that the patient undergo a treatment indicated in Table 1 under “Panel Results Indicate Inflammation” and
(b) if the expression levels do not indicate inflammation according to Table 1 or measurement of 80 HdG levels, recommending that the patient undergo treatment indicated in Table 1 under “Panel Results Do Not Indicate Inflammation”.

In additional embodiments,
(a) if the expression levels indicate inflammation according to Table 1 or measurement of 80 HdG levels, treating the patient with a treatment that reduces inflammation and
(b) if the expression levels do not indicate inflammation according to Table 1 or measurement of 80 HdG levels, treating the patient with a treatment that does not reduce inflammation.

In these embodiments, expression levels can be determined by (a) determining mRNA levels in a sample from the patient, or by (b) determining protein levels in the sample.

These determinations can be made by any means known in the art. In some embodiments, mRNA levels are determined by a next generation sequencing method, e.g., pyrosequencing, sequencing by reversible terminator, sequencing-by-ligation mediated by ligase enzymes, or real-time sequencing using phospholinked fluorescent nucleotides, etc. See, e.g., www.sciencedirect.com/topics/medicine-and-dentistry/next-generation-sequencing. Examples of Table 1 biomarkers that can be determined by determining mRNA levels are ABCA1, ADAMTS4, CACNA1C, CASP1, CASP3, CASP9, CCL27, CRHR1, EDAR, EPO, FGFR2, FKBP5, GCKR, GH1, HAWP, HFE, HFE2, GF1, IL-1, 11-10, 1L-18, IL-11B, 1L-6, KRT1, KRT83, STK11, MMP13, MUC1, NFKB1, NFKB2, NLRP3, NLRP1, NR3C1, PAX8, PDL1, PPARG, RGS2, SDK1, SIRT1, SKA2, SLC11A2, SLC22A4, SLC40A1, SLC6A3, SMAD3, TGFB1, TNF, VEGFA, th-8, IL-17, TNF-α, TGF-β1, CASP-8, CASP-10, CASP-1, or TRAIL.

In other embodiments, protein levels are determined by immunological methods, e.g., enzyme-linked immunosorbent assay (ELISA). See, e.g., en.wikipedia.org/wiki/ELISA. Some of the proteins provided in Table 1 that can be determined by ELISA are described in Table 2.

TABLE 2
Protein biomarkers that can be measured by ELISA technology

| Proteins | Significance |
|---|---|
| Interleukin-1 (IL-1) | IL-1 is important in both the inflammatory response to cell injury and |
| | in homeostasis of cells, tissues, and organs. |
| Interleukin-6 (IL-6) | IL-6 is a protein that is produced by cells in the body in response to |
| | inflammation. It has been shown to be involved in tumor progression, |
| | including inhibition of cancer cell apoptosis. |
| Interleukin-8 (IL-8) | IL-8 helps maintain cancer cells in an embryonic state, which confers |
| | motility and invasiveness, and promotes angiogenesis. |
| Interleukin-10 | IL-10 acts as an anti-inflammatory and maintains the balance of the |
| (IL-10) | immune response to infection, allowing the clearance of pathogens |
| | while minimizing damage to host cells. |
| Interleukin-17 | IL-17 is a proinflammatory cytokine responsible for cell-mediated |
| (IL-17) | immunity against bacteria and fungi. |
| Interleukin-18 | IL-18 is a cytokine that regulates the immune system and predicts |
| (IL-18) | cardiovascular death rates in patients with stable and unstable angina. |
| Tumor necrosis | TNF has multiple immunomodulatory functions. It regulates the |
| factor-α | function of immune cells and induces fever, apoptosis, cachexia |
| (TNF-α) | (wasting syndrome), inflammation and sepsis. It inhibits tumorigenesis |
| | and viral replication and responds to sepsis by acting on |
| | monocytes/macrophages. |
| Transforming growth | TGF-β1 is a multifunctional protein that plays an essential role in many |
| factor-β (TGF-β1) | cellular processes, including the regulation of cell growth and |
| | proliferation, differentiation, and apoptosis. |
| Caspase 8 | The protein caspase-8 is critical to regulating apoptosis (programmed |
| | cell death), the main function of which is to promote cell death and |
| | eliminate auto aggressive T-cell clones that, if not eliminated, could |
| | contribute to the development of autoimmune diseases. |
| Caspase 10 | Caspase-10 is a protein found in the human body that acts as a negative |
| | regulator, inhibiting cell death and assisting with the induction of genes. |
| | It is important in preventing cancer from developing on the DNA level. |
| Caspase 1 | Caspase-1 activation is important in regulating immune cell response |
| | against microbial infections and metabolic activities, such as |
| | pyroptosis, a programmed cell death process. Thus, it is evident that |
| | inflammasomes play an important role in maintaining homeostasis in |
| | human body. |
| TRAIL | TRAIL is a cytokine that causes apoptosis primarily in tumor cells, by |
| | binding to certain death receptors. TRAIL is expressed in low levels in |
| | many healthy tissues, including the heart, kidney and lung; however, it |
| | is expressed at high levels in various tumors. The protein has been |
| | shown to induce tumor cell death without damaging surrounding |
| | tissues. |

Another biomarker that can be determined by ELISA is 80 HdG, discussed above.

Expression levels of the biomarker can be determined in any appropriate tissue, for example blood, urine, cells, or any tissue, for example a tissue that exhibits signs of the disease.

Any treatment, now known or later discovered, that (i) reduces inflammation, if the biomarker expression levels indicate inflammation, or (ii) does not reduce inflammation, if the biomarker expression levels do not indicate inflammation, can be recommended or administered to the patient. In some embodiments,
(a) if the expression levels indicate inflammation according to Table 1, treating the patient with a treatment indicated in Table 1 under “Panel Results Indicate Inflammation” and
(b) if the expression levels do not indicate inflammation according to Table 1, treating the patient with a treatment indicated in Table 1 under “Panel Results Do Not Indicate Inflammation”

Also provided is a method of evaluating patient response to a treatment for a chronic inflammatory disease. The method comprises
a) determining expression of at least one or more genes or protein biomarkers listed in Table 1 or measurement of 80 HdG levels in a population of subjects without the chronic inflammatory disease;
b) determining expression in the patient of the at least one or more genes or protein biomarkers listed in Table 1 or measurement of 80 HdG levels prior to the treatment; and
c) determining expression of the at least one or more genes or protein biomarkers after the treatment,
wherein i) the patient is responding to the treatment if expression of the at least one or more genes or protein biomarkers approaches the expression in a) after treatment, and
ii) the patient is not responding to the treatment if expression of the at least one or more genes or protein biomarkers does not approach the expression in a) after treatment.

In some embodiments, the one or more genes or protein biomarkers are ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, GF1, IL-1, IL-10, IL-18, IL-1B, IL-6, KRT1, KRT83, STK11, MMP13, MUC1, NFKB1, NFKB2, NLRP3, NLRP1, NR3C1, PAX8, PDL1, PPARG, RGS2, SDK1, SIRT1, SK A2, SLC11 A2, SLC22A4, SLC40A1, SLC6A3, SMAD3, TGFB1, TNF, VEGFA, IL-8, IL-17, TNF-α, TGF-β1, CASP-8, CASP-10, CASP-1, TRAIL, 80 HdG or any combination thereof.

In other embodiments, the disease is preeclampsia, bladder cancer, breast cancer, colon cancer, or a cardiac disease.

In additional embodiments, the disease is bladder cancer, breast cancer, or colon cancer and the one or more genes or protein biomarkers are KRT1, CCL27, SDK1, PPARG, ILIA, SMAD3, SLC6A3, NR3C1, KRT83, HFE2, CASP3, CACNA1C, NFKB2, CASP9, or any combination thereof. In some of these embodiments, the one or more genes or protein biomarkers are SLC6A3, NR3C1, KRT1, KRT83, HFE2, CCL27, or any combination thereof.

In further embodiments, the disease is breast cancer or colon cancer and the one or more genes or protein biomarkers are KRT1, CCL27, SDK1, PPARG or any combination thereof. In more specific embodiments, the one or more genes or protein biomarkers are KRT1, CCL27 or the combination thereof.

In other embodiments, the disease is bladder cancer or colon cancer and the one or more genes or protein biomarkers are IL1A, SMAD3 or the combination thereof.

In additional embodiments, the disease is bladder cancer or breast cancer and the one or more genes or protein biomarkers are ILIA, CCL27 or the combination thereof.

In some aspects of these methods, if the determinations indicate that the patient is responding to the treatment, no change in treatment is recommended. Conversely, if the determinations indicate that the patient is not responding to the treatment, then a change in treatment is recommended or implemented.

The present invention is also directed to a method of developing a treatment for a chronic inflammatory disease in a patient. The method comprises
(a) obtaining a potential treatment medication;
(b) determining expression levels in the patient of one or more genes or protein biomarkers listed in Table 1 or measurement of 80 HdG levels prior to administering the medication;
(c) administering the medication to the patient;
(d) determining expression levels in the patient of the one or more genes or protein biomarkers after administering the medication; and
(e) establishing the medication as a treatment for the disease if a change in expression levels between step (b) and step (d) indicates that the medication reduces or increases inflammation.

This method can be used to develop any treatment for any chronic inflammatory disease, e.g., as previously listed. In some embodiments, the chronic inflammatory disease is preeclampsia, bladder cancer, breast cancer, colon cancer, or a cardiac disease.

Expression levels of the biomarker can be determined in any appropriate tissue, for example blood, urine, cells, or any tissue, for example a tissue that exhibits signs of the disease.

In some of these embodiments, the expression levels are determined by determining mRNA levels of the one or more genes or protein biomarkers in blood, urine, cells, or a tissue of the patient. Nonlimiting examples of biomarkers that can be determined with mRNA and Elisa protein measurements levels include ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, GF1, IL-1, IL-10, IL-18, IL-1B, IL-6, KRT1, KRT83, STK11, MMP13, MUC1, NFKB1, NFKB2, NLRP3, NLRP1, NR3CI, PAX8, PDL1, PPARG, RGS2, SDK1, SIRT1, SKA2, SLC11A2, SLC22A4, SLC40A1, SLC6A3, SMAD3, TGFB1, TNF, VEGFA, IL-8, IL-17, TNF-α, TGF-β1, CASP-8, CASP-10, CASP-1, TRAIL, 80 HdG or any combination thereof.

In other embodiments, the expression levels are determined by determining levels of the protein encoded by the one or more genes or protein biomarkers in a tissue of the patient. Nonlimiting examples of biomarkers that can be determined by determining levels of the protein include Interleukin1 (IL-1), Interleukin-6 (IL-6), Interleukin-8 (IL-8), Interleukin-10 (IL-10), Interleukin-17 (IL-17), Interleukin-18 (IL-18), Tumor necrosis factor-α (TNF-α), Transforming growth factor-β (TGF-β 1), Caspase 8, Caspase 10, Caspase 1, TRAIL, 80 HdG or any combination thereof.

These methods can also be used for determining treatments for any disease, now known or later discovered, that has an inflammatory component. Nonlimiting examples include a cancer (for example bladder cancer, breast cancer and colon cancer), preeclampsia, a cardiac disease (e.g., atherosclerosis or vasculitis), encephalitis, myelitis, meningitis, arachnoiditis, PNS, neuritis, dacryoadenitis, scleritis, episcleritis, keratitis, retinitis, chorioretinitis, blepharitis, conjunctivitis, uveitis, otitis externa, otitis media, labyrinthitis, mastoiditis, carditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis, osteitis/osteomyelitis, spondylitis, periostitis, chondritis, nephritis, glomerulonephritis, pyelonephritis, ureteritis, cystitis, urethritis, oophoritis, salpingitis, endometritis, parametritis, cervicitis, vaginitis, vulvitis, mastitis, orchitis, epididymitis, prostatitis, seminal vesiculitis, balanitis, posthitis, balanoposthitis, chorioamnionitis, funisitis, omphalitis, insulitis, hypophysitis, thyroiditis, parathyroiditis, adrenalitis, lymphangitis and lymphadenitis.

In some embodiments, the disease is preeclampsia, bladder cancer, breast cancer, colon (e.g., colorectal) cancer, or a cardiac disease, e.g., atherosclerosis or vasculitis.

In additional embodiments, the disease is also diagnosed, monitored or prognosed by determining whether genes are up regulated or down regulated in accordance with Table 1.

### Colorectal Cancer

Colorectal cancer (CRC) is the third most common cancer in the world, which accounted for almost 1.4 million new cases and 694,000 deaths in 2012. No single predominant risk factor attributes to CRC, but family history of CRC, smoking, excessive alcohol consumption, high intake of red and processed meat, obesity, physical inactivity and diabetes have been identified as risk factors, apart from age and male sex, in epidemiologic studies.

Inflammatory bowel disease, such as ulcerative colitis and Crohn's disease, is an established risk factor for CRC, suggesting link of chronic inflammation with malignant disease. This is also supported by lowered risk associated with regular use of aspirin and other non-steroidal anti-inflammatory drugs (NSAIDs), seen in observational studies and randomized clinical trials, and in studies reporting that polymorphisms in inflammation genes are associated with CRC risk. (Song L, et al. 2018. Circulating Inflammatory Markers and Colorectal Cancer Risk: A Prospective Case-cohort Study in Japan. Int J Cancer. 143(11): 2767-2776.)

“Among the six markers found to be significantly associated with CRC risk, five were chemokines. Chemokines were originally identified as a class of cytokines that act as potent attractants for white blood cells to sites of infection or injury, acting as mediators of acute and chronic inflammation.”

“With regards to carcinogenesis, chemokines may recruit tumor-associated leukocytes which induce angiogenesis and control tumor cell invasion, or directly induce migration of epithelial/endothelial cells that express their receptors.” Id.

The following genes are relevant with colorectal cancer (see also Example 3):

### KRT1 (Keratin 1)

Keratin 1 maintains the intestinal barrier by upregulating tight junction proteins in ulcerative colitis. (Wu, J. et al. 2021. Keratin I maintains the intestinal barrier in ulcerative colitis. Genes Genomics. 43(12):1389-1402).

“KRT1 was presumed to play some roles of maintaining intestinal epithelium barrier and the progression of inflammatory bowel disease (IBD). In this study, we found that the expression of KRT1 was detected in intestinal tissue and its down regulation was associated with the progression of IBD. In addition, KRT1 played an important role in maintaining epithelial barrier during a challenge of interleukin 1-beta (IL-1p).” (Dong, X. et al. 2017. Critical role of Keratin 1 in maintaining epithelial barrier and correlation of its down regulation with the progression of inflammatory bowel disease. Gene. 608: 13-19.)

### SDK1

Barrow and colleagues performed an epigenome-wide analysis of DNA methylation in colorectal cancer (CRC) patients with different smoking statuses, and among the significant hypomethylated genes were the SDK1, PTPRN2, and TNXB genes. (Barrow T. M., et al. 2017. Smoking Is Associated with Hypermethylation of the APC 1A Promoter in Colorectal Cancer: The ColoCare Study. J. Pathol. 243:366-375.)

“In our study, we identified 116 hypomethylated SDK1 loci and 83 hypomethylated loci in each of the PTPRN2 and TNXB genes. These three genes also contained the highest number of hypomethylated loci.” (Baharudin, R. et al. 2022. Epigenome-Wide DNA Methylation Profiling in Colorectal Cancer and Normal Adjacent Colon Using Infinium Human Methylation 450K. Diagnostics (Basel). 12(1): 198.)

Sidekick cell adhesion molecule 1 (SDK1) hypomethylation was also reported in sporadic colorectal cancer. (Farkas S. A., et al. 2014. DNA Methylation Changes in Genes Frequently Mutated in Sporadic Colorectal Cancer and in the DNA Repair and Wnt/β-Catenin Signaling Pathway Genes. Epigenomics. 6:179-191.)

### CASP9 (Caspase 9)

Caspase 3, 8 and 9 are important molecules in apoptotic (programmed cell death) pathways which play key roles in cancer development and progression. (Fearnhead H O, et al. 1998. Oncogene-dependent apoptosis is mediated by caspase-9. Proc Natl Acad Sci USA. 95:13664-9.) See also Bodmer W., et al. 1994. Genetic steps in colorectal cancer. Nat Genet. 6:217-9 (Achieving approaches toward prevention and early diagnosis of CRC requires better understanding of genetic and molecular pathways involved in the disease etiopathogenesis.)

“Due to important functions of caspase proteins in cancers, we evaluated expression levels of them in CRC tissues and compared them with normal marginal tissues. It was also aimed to reveal if there was any relation between mRNA expression level of these molecules and clinical features of the patients.”

“Caspase 9 expression level was downmodulated in tumoral tissues compared with normal marginal colon samples, which had previously been shown by other studies (Fearnhead et al., 1998 Id.). Moreover, association of mRNA expression level of caspase 9 and clinical manifestations of CRC patients was found in this study, as it was in accordance with another study (Shen et al., 2010. Downregulation of caspase-9 is a frequent event in patients with stage II colorectal cancer and correlates with poor clinical outcome. Colorectal Dis. 12:1213-8.)

“In conclusion, modifications in molecular markers throughout the onset and progression of malignancies can underpin the designing of much effective therapeutics and diagnostic tools and may prevent cancer development in early diagnosed cases. These molecular signatures can then be applied as a target of novel therapeutic strategies. In the ongoing investigation, we observed an aberrant mRNA expression level of caspase 3, 8, and 9 in tumoral tissues of CRC patients in relation to normal marginal tissues. Furthermore, the altered expression level of these genes was related to some of clinicopathological specifications of the patients. It is essential to perform further studies to confirm caspase molecules as therapeutic or diagnostic tool in CRC patients.” (Asadi M, et al. 2018. Expression Level of Caspase Genes in Colorectal Cancer. Asian Pac J Cancer Prev. 19(5): 1277-1280.)

### CCL27

“Given their pleiotropic activity, increased levels of chemokines could either promote or inhibit carcinogenesis. We found elevated levels of 3 CC chemokines (CCL2/MCP1, CCL15/MIP1D and CCL27/CTACK) and decreased level of 1 CC chemokine (CCL3/MIP1A) and 1 CXCL chemokine (CXCL6/GCP2) to be associated with increased CRC risk.” Song L, et al. 2018 Id. See also Example 3.

Based in part on Example 3, the present invention is also directed to a method of treating a cancer in a patient. The method comprises
a) determining whether the patient has bladder cancer, colon cancer, or breast cancer by
   i) measuring an mRNA level of at least one gene in a tissue of the patient, wherein the at least one gene is KRT1, CCL27, SDK1, PPARG, ILIA, SMAD3, SLC6A3, NR3C1, KRT83, HFE2, CASP3, CACNA1C, NFKB2, CASP9, or any combination thereof;
   ii) comparing the mRNA levels determined in i) with an mRNA level of the at least one gene from a reference tissue from
      A) at least one subject with bladder cancer, and/or B) at least one subject with colon cancer, and/or C) at least one subject with breast cancer; and
   iii) treating the patient for bladder cancer if the mRNA level(s) determined in i) is similar to the mRNA level from ii)A), or treating the patient for colon cancer if the mRNA level(s) determined in i) is similar to the mRNA level from ii)B), or treating the patient for breast cancer if the mRNA level(s) determined in i) is similar to the mRNA level from ii)C).

In some embodiments, the at least one gene is KRT1, CCL27, SDK1, PPARG or any combination thereof, and the method determines whether the patient has breast cancer or colon cancer. In some of these embodiments, the at least one gene is KRT1, CCL27 or the combination thereof.

In other embodiments, the at least one gene is IL1 A, SMAD3 or the combination thereof, and the method determines whether the patient has bladder cancer or colon cancer.

In additional embodiments, the at least one gene is IL1A, CCL27 or the combination thereof, and the method determines whether the patient has bladder cancer or breast cancer.

In further embodiments, the at least one gene is SLC6A3, NR3C1, KRT1, KRT83, HFE2, CCL27, IL1A, SMAD3, CASP3, SDK1, CACNA1C, NFKB2, PPARG, CASP9, or any combination thereof, and the method determines whether the patient has bladder cancer, breast cancer or colon cancer. In some of these embodiments, the at least one gene is SLC6A3, NR3C1, KRT1, KRT83, HFE2, CCL27, or any combination thereof.

Any biologic fluid or tissue from the patient that contains mRNA can be utilized in these methods, for example saliva, blood or any mRNA-containing portion thereof (e.g., serum, plasma, lymphocytes, exosomes, etc.), urine, feces, swab of buccal cheek cells, a biopsy from any organ, etc. In some embodiments, the tissue sample is a swab of buccal cheek cells.

### Role of Nutritional Supplements for Treating Inflammatory Diseases

In some embodiments, the treatment includes administration of a nutritional supplement.

Any discussion involving the topic of inflammation must also explain the relationship of inflammation to oxidative stress and immune system function. Oxidative stress can occur when there is an imbalance of free radicals and antioxidants in the body. Keep in mind that normal body function produces free radicals during normal metabolic processes. Like a car engine burning fuel, in this case, the fuel is food. To counteract these free radicals, cells naturally produce antioxidants. In normal health, the body maintains a balance between antioxidants and free radicals. When the immune system responds to infection or injury, it also triggers a temporary oxidative stress response. Normally, inflammation goes away after the immune system eliminates the infection or repairs the damaged tissues. When the body is overwhelmed with an out of control inflammatory or oxidative stress situation, chronic disease can develop, such as diabetes, heart disease and arthritis, to name a few. Another example that we are faced with during the current Covid-19 pandemic is a viral induced pneumonia associated with excessive production of inflammatory cytokines. This reaction, known as cytokine storm, can be a life-threatening complication (Monteleone, 2020)

This inflammatory or oxidative stress process is especially evident in the immune cells, which use free radicals in their functions and suffer a senescent deterioration probably linked to oxygen stress. “Therefore, since the immune system is an indicator of health and a longevity predictor, the protection of this system afforded by dietary antioxidant supplementation may play an important role in order to achieve a healthy ageing.” (Fuente, 2002)

Inflammation is like a light switch which can be turned on and off; The Problem: It can stay on and cause both silent and visible disease. Example: Cancer is an inflammatory wound “that does not heal” (Byun, 2013).

The ability to switch the out of control ‘inflammatory light’ off is the answer. The only problem is that there is no light or beacon that tells us inflammation has occurred within our body. Chronic inflammation is a silent disorder. All people are being bombarded daily with chronic inflammation which is slowly waging a war of attrition on our tissues and organs. Picture air and water pollution, ionizing radiation from the sun, smoking, automobile emissions, etc.

Most doctors only focus on observable inflammatory disease and symptoms in specific organs while the rest of the body may be silently deteriorating from the same insidious inflammatory process.

How does one prevent out of control inflammation? Identify the genes that control inflammation and the release of the causative cytokine signaling molecules. (Natoli, 2011).

Prevention is the rule of the day! Not intervention which is the basic philosophy of the pharmaceutical industry. It was the great physician Hippocrates, who in 450 BC, stated “Let Food Be Thy Medicine and Medicine Be Thy Food.” This quote rings true even today as we face one infectious insult after another—SARS, Ebola, COVID-19, etc. “Although many patients are convinced of the importance of food in both causing and relieving their problems, many doctors' knowledge of nutrition is rudimentary” (Smith, 2004) A preventive natural nutritional anti-inflammatory formulation, NuGenea™, has been designed that utilizes compounds that have the potential to modulate expression of the genes that produce the inflammatory molecules that cause inflammation.

“Therefore, the role that nutritional components, in particular vitamins and minerals, play in the modulation of miRNA profiles, and consequently health and disease, is increasingly being investigated, and as such is a timely subject for review. The recently posited potential for viable exogenous miRNA to enter human blood circulation from food sources adds another interesting dimension to the potential for dietary miRNA to contribute to gene modulation” (Beckett, 2014).

The current healthcare climate is opportune for the introduction of a proprietary well-designed nutritional supplement formula in which the possible effect of the whole is better than any of the parts. The NuGenea™ nutritional formulation is an attempt to integrate nutrients that may be complementary with each other with the potential objective of supporting the immune system and modulating chronic inflammation while it is still a silent disorder. Example: vitamin A plus zinc may reduce the risk of developing advanced age-related macular degeneration by about 25 percent; see NuGenea™ descriptive discussion for vitamin A below.

The objective is for a person to build up an internal storehouse of these important anti-inflammatory compounds while one is healthy. When disease hits, your body can call up these needed compounds to naturally enhance the immune system and neutralize the out of control inflammatory processes. People during the current Coronavirus pandemic are stocking up and hoarding toilet paper, drugs, and even dried food with 25-year storage capacity. The same idea/picture holds true for one's body and immune system. What is the use of all these external products when your own ‘internal system’ is being attacked and decimated?

An example of inflammation potentially out of control is the formation of blood clots in veins, called venous thromboembolism (VTE). 40% of people who develop VTE do not know what caused it, yet it is the third deadliest cardiovascular diagnosis. “We don't yet understand the molecular triggers which drive the development of life-threatening clots in deep veins,” said Yogen Kanthi, M. D., the study's senior author and a vascular cardiologist at U-M's Frankel Cardiovascular Center. “Our work aimed to identify and block a previously unrecognized pathway linking inflammation and thrombosis.” Kanthi's team found an enzyme called CD39 diffused circulating “danger” signals and inflammatory cytokines in blood during thrombosis (Anyanwu, 2019).

The ability to fight disease is like fighting crime; when there are more cops on the street, more criminals are arrested. A commonsense approach is to build up one's health reserve through a well-rounded nutritional formula that places into storage the nutritional ammunition that will be called up to fight disease when it raises its ugly head.

The focus of the unique NuGenea™ formula of ingredients is the potential modulation and/or reduction of the inflammatory genetic markers associated with chronic disease.

The substances in the formulation are listed individually and the application to the inflammatory process is described below. Scientific articles supporting these statements are shown. However, keep in mind that many nutritional related clinical studies are in animals. The hurdles that clinical nutrition researchers encounter is well stated in an article by Weaver and Miller. “Clinical nutrition researchers encounter many hurdles, including difficulties with recruiting volunteers, navigating a complex maze of approvals, and coping with myriad biases.” “Special scientific issues involved with clinical nutrition research include study designs that increase or decrease the status of a nutrient, food, or bioactive reagent but often do not compare presence with absence of the compound (as is typical in drug trials); ethical issues regarding withholding of a nutrient from participants who are low or deficient in that nutrient; study populations that may already be sufficient in the compound of interest and, thus, may not show benefit of supplementation; interventions that are difficult to blind to both the subjects and the investigators; and a tension between studying subgroups most likely to respond versus recruiting a representative and, therefore, generalizable sample.” (Weaver, 2017).

Be your own advocate in the current healthcare environment. Let your body be your guide! Nature is all around us. The pharmaceutical industry is very young, maybe only 200 years old. Life has been around a lot longer! Do we have the liberty to wait to protect our health? Time is the enemy and is not our friend.

### Components—NuGenea™

### 1. Alpha Lipoic Acid (ALA)

ALA is described as a direct antioxidant and anti-inflammatory agent. Iron is a heavy metal which is important in carrying oxygen within the red blood cell and is also a cause of oxidative and inflammatory damage. ALA has the potential to inhibit iron-mediated oxidative damage. Industry Claimed Uses: cognitive impairment, weight management, nerve disorders (neuropathy, MS), diabetes, atherosclerosis (endothelial syndrome), liver disease and lipid related disorders. Science Related Genetic Application: upregulates nuclear factorE2 (Nrf2) dependent pathway. Codes for inflammatory mediator such as superoxide dismutase (SOD).

### Scientific Articles:

a. Linus Pauling Institute; Oregon State University. Nutrition/Supplements/Lipoic Acid. Linus Pauling Institute. Oregon State University.
b. Salehi, B. et al. 2019. Insights on the Use of α-Lipoic Acid for Therapeutic Purposes. Biomolecules. 9(8): 356.

### 2. N-Acetyl Cysteine (NAC)

NAC is a powerful free radical scavenger, especially oxygen radicals involved in uncontrolled inflammation. Powerful antioxidant. Described as safe and well-tolerated. Involved in glutathione synthesis.
Industry Claimed Uses: chronic bronchitis, ulcerative colitis, intestinal dysfunction, muscle performance, asthma, Parkinson's disease, etc.
Science Related Genetic Application: reduced TNF-α and NF-κB, IFN-γ, and 1L-6 expression in LPS-induced cells in the pig small intestine (Lee, 2019)

### Scientific Articles:

a. Review article; Mokhtari et al. A Review on Various Uses of N-Acetyl Cysteine. 2017; Cell Journal, Vol 19, No 1, 11-17.
b. Salamon, S. et al. Medical and Dietary Uses of N-Acetylcysteine. 2019 Antioxidants, 8, 111.
c. Lee, S I and Kang, KS. N-acetylcysteine modulates lipopolysaccharide- induced intestinal dysfunction.
d. Liao, C Y et al. Protective effect of N-acetylcysteine on progression to end-stage renal disease: Necessity for prospective clinical trial. 2019. Europ. J. Int. Med. 44,67-73.
e. Repine, J E et al. Oxidative Stress in Chronic Obstructive Pulmonary Disease (The Oxidative Stress Study Group). 1997. Am J Respir Crit Care Med Vol. 156. pp. 341-357.

### 3. Zinc Picolinate

Zinc is an essential mineral with antioxidant and anti-inflammatory properties and plays an important role in cell mediated immunity. Supplementation studies in the elderly with this essential micronutrient have shown decreased incidence of infections, decreased oxidative stress, and decreased generation of inflammatory cytokines.

Industry Claimed Uses: prevent free-radical induced tissue injury inflammatory reactions, enhance immune function, stabilize blood sugar levels, promote healthy skin, eyes and heart.

Science Related Genetic Application: Zinc is involved in the modulation of the proinflammatory response by targeting Nuclear Factor Kappa B (NF-κB), a transcription factor that is the master regulator of proinflammatory responses. In vitro studies have shown that zinc decreases NF-κB activation and its target genes, such as TNF-α and IL-1β, and increases the gene expression of A20 and PPAR-α, the two zinc finger proteins with anti-inflammatory properties.

“After zinc supplementation, the incidence of infections was significantly lower, plasma zinc was significantly higher, and generation of tumor necrosis factor and oxidative stress markers was significantly lower in the zinc-supplemented than in the placebo group.” (Prasad 2007).

A study by the Age-Related Eye Disease Study Research Group (AREDS), Albany, NY, revealed that zinc had the potential when combined with other antioxidants to reduce the odds of developing visual loss in age-related macular degeneration (AMD).

### Scientific Articles:

a. AREDS Report #8. A Randomized, Placebo-Controlled, Clinical Trial of High-Dose Supplementation with Vitamins C and E, Beta Carotene, and Zinc for Age-Related Macular Degeneration and Vision Loss. 2001. Arch Ophthalmol. 119(10):1417-1436.
b. Jarosz, M. et al. 2017. Antioxidant and anti-inflammatory effects of zinc. Zinc-dependent NF-κB signaling. Inflammopharmacology. 25(1): 11-24.
c. Prasad, A S et al. Zinc supplementation decreases incidence of infections in the elderly: effect of zinc on generation of cytokines and oxidative stress. 2007. Am J Clin Nutr. 85:837-44.
d. Prasad, AN. Zinc in Human Health: Effect of Zinc on Immune Cells. 2008. Mol. Med. 14(5-6): 353-357.

### 4. Coenzyme Q10 (CoQ10)

Coenzyme Q10 (CoQ10) is an antioxidant that your body produces naturally. Human cells use CoQ10 for growth and maintenance. Levels of CoQ10 in your body decrease as you age. CoQ10 levels have also been found to be lower in people with certain conditions, such as heart disease (Mayo Clinic, 2017).

Industry Claimed Uses: heart conditions, Parkinson' disease, muscle weakness associated with use of statins, migraines, cellular energy production, physical performance.

Science Related Genetic Application: Clinical study showed reduction in the inflammatory cytokine TNF-α in rheumatoid arthritis patients. There was no significant difference in IL-6 levels between control and treated groups. (Abdollahzad, 2015). CoQ10 supplementation reduced inflammatory markers (TNF-α, IL-6, and MMP-9) in patients with multiple sclerosis (MS) (Sanoobar, 2015).

### Scientific Articles:

a. Abdollahzad, H. et al. 2015. Effects of Coenzyme
b. Q10 Supplementation on Inflammatory Cytokines (TNF-α, IL-6) and Oxidative Stress in Rheumatoid Arthritis Patients: A Randomized Controlled Trial. Archives of Medical Research. 46:7, 527-533.
c. Sanoobar, M. et al. 2015. Coenzyme Q10 supplementation ameliorates inflammatory markers in patients with multiple sclerosis; a double blind, placebo, controlled randomized clinical trial. Nutr. Neuroscience. 18(4): 169-76.
d. Zhai, J et al. 2017. Effects of Coenzyme Q10 on Markers of Inflammation: A Systematic Review and Meta-Analysis. PLOS ONE|DOI: 10.1371/journal.pone.0170172.
e. https://www.mayoclinic.org/drugs-supplements-coenzyme-q10/art- 20362602).
f. https://www.nccih.nih.gov/health/coenzyme-q10.

### 5. Glutathione (GSH):

Humans synthesize glutathione and this antioxidant is found throughout the structure of cells. GSH is capable of preventing damage to important cellular components caused by reactive oxygen species such as free radicals, peroxides, lipid peroxides, and heavy metals.

Industry Claimed Uses: Cataracts and glaucoma, asthma, heart disease (high cholesterol), cancer, memory loss, fatigue, arthritis, etc. There are opinions that GSH can reverse the aging process.

Science Related Genetic Application: “GSH is not just an inhibitor of inflammation, but it is required to allow a proper response to infection, and “direct” the migration of inflammatory cells (PMN) away from the lung, where they cause acute respiratory disease syndrome (ARDS), and towards the site of infection, where they kill bacteria.” “This essential role of GSH in immunity might explain why in many diseases, not only AIDS, decreased GSH levels are associated with an increased susceptibility to infection. These include COPD, cystic fibrosis, influenza infection, and alcoholism, as ethanol impairs Th1/Th2 balance via GSH depletion.” (Ghezzi, 2011). Increased oxidative stress as reflected by decreased GSH was associated with a decline in executive function (cognitive decline) in a healthy population. This study demonstrates that decreased circulating levels of glutathione predict the age-related decline in the executive domain, an area of cognition that is particularly susceptible to cardiovascular disease. (Hajjar, 2018).

### Scientific Articles:

a. Ghezzi, P. 2011. Role of glutathione in immunity and inflammation in the lung. International Journal of General Medicine 4: 105-113.
b. Hajjar, 1 et al. 2018. Oxidative stress predicts cognitive decline with aging in healthy adults: an observational study. Journal of Neuroinflammation (2018) 15:17.
c. Vitamins: “A vitamin is a substance that makes you ill if you don't eat it.” (Albert Szent-Gyorgyi, Nobel Prize in Physiology or Medicine, 1937).

### 6. Vitamin A:

Vitamin A is necessary for normal differentiation of epithelial tissues, the visual process and reproduction, and is vital for the optimal maintenance and functioning of the innate and adaptive immune system. Vitamin A deficiency is one of the most profuse nutritional deficiencies worldwide. It is associated with increased susceptibility to infectious diseases in both man and animal models. Vitamin A also has a role as an anti-inflammatory agent. Supplementation with vitamin A has been found to be beneficial in a number of inflammatory conditions, including skin disorders such as acne vulgaris, broncho-pulmonary dysplasia and some forms of precancerous and cancer states. The present review suggests that vitamin A deficiency induces inflammation and aggravates existing inflammatory states. Supplementation with vitamin A in selected cases could ameliorate inflammation. The two main mechanisms which appear to be involved in the prevention of disease are the effects of vitamin A on the immune system and the effect on epithelial integrity (Reifen, 2002).

Industry Claimed Uses: Healthy vision, skin, bones and other tissues. Antioxidant fighting cell damage.

Science Related Genetic Application: “The drastic reduction in the levels of IGF-I mRNA and the resulting decrease in serum IGF-I concentrations may well explain the growth retardation in vitamin A-deficient animals. To our knowledge, this is the first in vivo report on the effects of vitamin A on IGF system components.” (Fu, 2001). “In children, vitamin A deficiency results in increased risks of mortality and morbidity from measles and diarrheal infections, blindness, and anemia, and among women it is likely to be associated with high mortality related to pregnancy. Many of these effects can be linked to the immunological functions of vitamin A” (Villamor, 2005). An age-related eye disease study by the National Eye Institute (NEI/NIH,2001), (AREDS, 2001), found that taking high levels of antioxidants, such as vitamin A, along with zinc, may reduce the risk of developing advanced age-related macular degeneration by about 25 percent.

### Scientific Articles:

a. Age-Related Eye Disease Study Research Group. A Randomized, Placebo-Controlled, Clinical Trial of High-Dose Supplementation with Vitamins C and E, Beta Carotene, and Zinc for Age-Related Cataract and Vision Loss: AREDS Report No. 9. Arch Ophthalmol. 2001 Oct.
b. Antioxidant Vitamins and Zinc Reduce Risk of Vision Loss from Age- Related Macular Degeneration. 2001. National Eye Institute. https://www.nei.nih.gov/about/news-and-events/news.
c. Fu, Z et al, 2001. Vitamin A Deficiency Reduces Insulin-Like Growth Factor (IGF)-I Gene Expression and Increases IGF-I Receptor and Insulin Receptor Gene Expression in Tissues of Japanese Quail (Coturnix coturnix japonica). J. Nutr. 131: 1189-1194.
d. Reifen, R. 2002. Review. Vitamin A as an Anti-Inflammatory Agent. Proc Nutr Soc. 61(3):397-400.
e. Villamor, E. and Fawzi, W W. 2005. Effects of Vitamin A Supplementation on Immune Responses and Correlation with Clinical Outcomes. Clin. Microb. Rev., July 2005, p. 446-464.

### 7. Vitamin C:

Vitamin C, also called ascorbic acid, plays a role in many bodily functions. According to the U.S. National Institutes of Health (NIH), the body uses vitamin C to make skin, tendons, ligaments, and blood vessels. It also uses this vitamin to repair and maintain cartilage, bones, and teeth, to heal wounds and to form scar tissue. Vitamin C may also prevent cancer by blocking the damage made by free radicals. Aids in the absorption of iron. With respect to its function as an anti- inflammatory compound, Vitamin C is a potent reducing agent, meaning that it readily donates electrons to recipient molecules. It is therefore a significant antioxidant and anti-inflammatory compound. Even in small amounts, vitamin C can protect indispensable molecules in the body, such as proteins, lipids (fats), carbohydrates, and nucleic acids (DNA and RNA), from damage by free radicals and reactive oxygen species (ROS) that are generated during normal metabolism, by active immune cells, and through exposure to toxins and pollutants (e.g., certain chemotherapy drugs and cigarette smoke). (Linus Pauling Institute).

Industry Claimed Uses: heart disease, atherosclerosis, diabetes, stroke, and cancer.

Science Related Genetic Application: Dietary supplementation with antioxidant vitamins, especially vitamin C resulted in a significant decrease in the mRNA expression of pro-inflammatory cytokines. Study done in chick animal model (Jang, 2014). Potential to modulate cell response when inflammation occurs (Canali, 2014). Vitamin C is necessary for the immune system to mount and sustain an adequate response against pathogens, whilst avoiding excessive damage to the host. Vitamin C appears to be able to both prevent and treat respiratory and systemic infections by enhancing various immune cell functions (Carr, 2017)

### Scientific Articles:

a. Canali, R et al. 2014.Vitamin C supplementation modulates gene expression in peripheral blood mononuclear cells specifically upon an inflammatory stimulus: a pilot study in healthy subjects. Genes Nutr. 9:390.
b. Carr, A C and Maggini, S. 2017. Vitamin C and Immune Function. Nutrients. 9 1211.
c. Jang, I et al. 2014. Effects of Vitamin C or E on the Pro-inflammatory Cytokines, Heat Shock Protein 70 and Antioxidant Status in Broiler Chicks under Summer Conditions. Asian Australas. J. Anim. Sci. 27:749-756.
d. Linus Pauling Institute, Oregon State University. https://lpi.oregonstate.edu/mic/vitamins/vitamin-C#function

### 8. Vitamin D

Vitamin D is fat-soluble vitamin that helps the body absorb and retain calcium and phosphorus which are both critical for building bone. Laboratory studies have shown the potential for this vitamin to reduce cancer cell growth, help control infections and reduce inflammation. Many of the body's organs and tissues have receptors for vitamin D, which suggest important roles beyond bone health. Cells of the immune system are capable of synthesizing and responding to vitamin D. Immune cells in autoimmune diseases are responsive to the ameliorative effects of vitamin D suggesting that the beneficial effects of supplementing vitamin D deficient individuals with autoimmune disease may extend beyond effects on bone and calcium homeostasis (Aranow, 2011).

Recently, increasing evidences have shown that the abnormal inflammatory response is closely associated with many chronic diseases, especially in autoimmune diseases, including rheumatoid arthritis (RA), inflammatory bowel disease (IBD), systemic lupus erythematosus (SLE), gout, and diabetes (Duan, 2019). This important regulatory function of vitamin D in autoimmune diseases and inflammation is emphasized by this statement of Yang, “impaired vitamin D signaling and/or inadequate vitamin D intake caused by genetic predisposition (e.g. VDR polymorphisms) and/or environmental factors (e.g. insufficient sunlight exposure in high-latitude regions or during the cold season) may contribute to the onset and progression of autoimmunity. Because of the high prevalence of vitamin D insufficiency/deficiency in patients with MS (sic. Multiple Sclerosis), T1DM, and SLE, vitamin D supplementation has been considered a prospective candidate for the treatment of such autoimmune diseases” (Yang, 2013).

Industry Claimed Uses: Bone health, cancer, heart disease, diabetes, and immune function.

Science Related Genetic Application: The actions of the vitamin D hormone 1,25-dihydroxyvitamin D3 (1,25(OH)2D3), the active compound, are mediated by the vitamin D receptor (VDR), which is located on many cells throughout the body. This active form of D plays a critical role in the control of gene expression (Pike, 2010). Importantly, vitamin D and the vitamin D receptor (VDR) which is present on most key cells of the body function in mRNA expression and regulation (Campbell, 2014). Potential to regulate cytokine production; if true, vitamin D can modulate the inflammatory process that is involved in tumor development and potentially also in viral pneumonia, such as cytokine storm (Liu, 2018). 1,25(OH)2VD3 can upregulate CCR10 on human T cells and ASCs while blocking the expression of skin- and gut-homing receptors. However, the in vivo relevance of the effects of 1,25(OH)2VD3 on CCR10 expression by T cells that are infiltrating the skin and by IgA+ASCs that are migrating to the gut lamina propria remains to be determined (Mora, 2008).

### Scientific Articles:

a. Aranow, C. 2011. Vitamin D and the immune system. J Investig Med. 2011 August; 59(6): 881-886.
b. Campbell, M J. 2014. Vitamin D and the RNA transcriptome: more than mRNA regulation. Front Physiol. 5: 181.
c. Duan, L. et al. 2019. Editorial: Regulation of Inflammation in Autoimmune Disease. Journal of Immunology Research Volume 2019, Article ID 7403796, 2 pages.
d. Pike, J W. And Meyer, M B. 2010. The Vitamin D Receptor: New Paradigms for the Regulation of Gene Expression by 1,25-Dihydroxyvitamin D3. Endocrinol Metab Clin North Am. 39(2): 255-269.
e. Liu, W. et al. 2018. The Anti-Inflammatory Effects of Vitamin D in Tumorigenesis. Int J Mol Sci. 19(9): 2736.
f. Mora, JR, Iwata, M and von Andrian, U H. 2008. Vitamin effects on the immune system: vitamins A and D take centre stage. Nat Rev Immunol. 8(9): 685-698.
g. The nutrition Source; Vitamin D. https://www.hsph.harvard.edu/nutritionsource/vitamin-d/
h. Yang, C Y et al, 2013. The Implication of Vitamin D and Autoimmunity: a Comprehensive Review. Clin Rev Allergy Immunol. 45(2): 217-226.
   9. Vitamin E; α-tocopherol

Vitamin E is not a single vitamin, but rather a group of fat-soluble vitamins. Its main role is to act as an antioxidant, scavenging loose electrons—so called “free radicals”—that can damage cells. Cardiovascular disease is the leading cause of morbidity and mortality in westernized populations. Low levels of α-tocopherol (AT) are associated with increased incidence of atherosclerosis and increased intakes appear to be protective (Devaraj, 1999). Vitamin E deficiency can cause nerve pain (neuropathy) (Staff, 2014).

Industry Claimed Uses: Research on its benefits is mixed. Oil used in cosmetics as moisturizer, wound healing, minimize healing scars after surgery, psoriasis.

Science Related Genetic Application: Vitamin E blocks the release of pro-inflammatory cytokines, including IL-1, IL-6, TNF, and the chemokine IL-8, by monocytes and macrophages (Devaraj, 1999). Moreover, vitamin E prevents the upregulation of the adhesion molecules vascular cell-adhesion molecule 1 (VCAM1) and intercellular adhesion molecule 1 (ICAM1) on the endothelium induced by oxidized low-density lipoprotein (LDL) and IL-10, as well as the upregulation of E-selectin and some chemokines. Reactive oxygen species activate the nuclear factor-κB (NF-κB) pathway, which initiates many pro-inflammatory events. Therefore, the therapeutic antioxidant effect of these vitamins could be explained, at least in part, by their capacity to decrease NF-κB activation (Mora, 2008).

### Scientific Articles:

a. Devaraj S, Jalal I. 1999. Alpha-tocopherol decreases interleukin-1 beta release from activated human monocytes by inhibition of 5-lipoxygenase. Arterioscler Thromb Vasc Biol. 19(4):1125-33. Arterioscler Thromb Vasc Biol. 1999 April; 19(4):1125-33.
b. Mora, JR, Iwata, M and von Andrian, U H. 2008. Vitamin effects on the immune system: vitamins A and D take centre stage. Nat Rev Immunol. 8(9): 685-698.
c. Munteanu A, Zingg J M. 2007. Cellular, molecular, and clinical aspects of vitamin E on atherosclerosis prevention. Mol Aspects Med. 28(5-6):538-90.
d. Staff, NP, Windebank, A J. 2014. Peripheral Neuropathy Due to Vitamin Deficiency, Toxins, and Medications. Continuum (Minneap Minn). 20(5):1293-1306.

### 10. Quercetin Dihydrate

One of the most abundant antioxidants in the diet and plays an important role in helping your body combat free radical damage and inflammation, which is linked to chronic diseases. Its antioxidant properties may help reduce inflammation, allergy symptoms, and blood pressure.

Industry Claimed Uses: Anti-inflammatory, ease allergy symptoms, cancer, blood pressure, exercise performance, maintain general health.

### Science Related Genetic Application:

A febrile seizure (FS) study in animals showed that IL-10 is genetically associated with FS and contrary to IL-1beta, confers resistance to FS (Ishizaki, 2009). Quercetin has anti-inflammatory properties and acts by suppressing pro-inflammatory cytokines (Comalada, 2005).
The mechanism by which quercetin increased IL-10 levels is not quite clear, but we suspect that it possibly triggered an early release of IL-10 to limit the activity of pro-inflammatory cytokines; this therefore suggests that it had neuroprotective effects (Nombuso, 2017). These findings, although not definitive, reveal the potential application of Quercetin to pneumonia associated with cytokine storm and febrile seizures.

### Scientific Articles:

a. Comalada M, et al. 2005. In vivo quercitrin anti-inflammatory effect involves release of quercetin, which inhibits inflammation through downregulation of the NF-kappaB pathway. Eur J Immunol. 35:584-592.
b. Ishizaki, Y. et al. 2009.Interleukin-10 is associated with resistance to febrile seizures: Genetic association and experimental animal studies. Epilepsia, 50(4):761-767.
c. Li, Y et al. 2016. Quercetin, Inflammation and Immunity. Nutrients 8, 167-181.
d. Nombuso, V P M et al. 2017. The Effect of Quercetin on Pro- and Anti-Inflammatory Cytokines in a Prenatally Stressed Rat Model of Febrile Seizures. J Exp Neurosci. 11:1-8.
e. Uttara, B et al. 2009. Oxidative Stress and Neurodegenerative Diseases: A Review of Upstream and Downstream Antioxidant Therapeutic Options. Current Neuropharmacology, 2009, 7, 65-74.

### 11. Alpha-GPC Choline

Choline is an organic, water-soluble compound. It is neither a vitamin nor a mineral; it is an essential nutrient found naturally in the brain. This essential nutrient has anti-inflammatory actions and data has shown its therapeutic potential in restraining excessive inflammation (Parris, 2006). Articles have been published discussing the potential therapeutic application of choline to treat diseases characterized by acute or chronic inflammation. One area of potential application is control of cytokine production; excessive cytokine release can lead to systemic inflammation, organ failure and death (Rosas-Ballina, 2009).

Industry Claimed Uses: DNA synthesis, healthy nervous system, supports structural integrity of cells.

Science Related Genetic Application: Cholinergic enhancing compounds act centrally in the body to decrease inflammation. In an experimental model of infection, called endotoxemia, substances that enhance levels of choline decrease serum biomarkers, TNF and IL-6 and improve survival of the animal model (Pavlov, 2008).

### Scientific Articles:

a. Parris, W R et al. 2006. Choline Suppresses Inflammatory Responses. Shock. 25 (6): 45.
b. Pavlov V A, Parrish W R, Rosas-Ballina M, et al. 2008. Brain acetylcholinesterase activity controls systemic cytokine levels through the cholinergic anti-inflammatory pathway. Brain Behav Immun. 23:41-5.
c. Rosas-Ballina, M and Tracey, KJ. 2009. Cholinergic Control of Inflammation. J Intern Med. 265(6): 663-679.

### 12. Resveratrol

Natural compound produced by several plants in response to injury or when the plant is attacked by pathogens, such as bacteria or fungi. It is a powerful antioxidant that protects a cell's DNA and helps prevent cell damage caused by free radicals. Free radicals are unstable atoms caused by pollution, sunlight and our bodies natural burning of fat that can lead to cancer, aging and brain degeneration.

Industry Claimed Uses: protection of the heart and circulatory system, lowering cholesterol, decreasing blood sugar, protection against blood clots.

Science Related Genetic Application: As a natural food ingredient, numerous studies have demonstrated that resveratrol possesses a very high antioxidant potential. Resveratrol also exhibits antitumor activity and is considered a potential candidate for prevention and treatment of several types of cancer. Some studies have also shown that this compound may act as a pro-oxidizing agent which may have implication in pathology of several diseases. Despite this double edge statement, the authors go on to state: “Resveratrol-like other derivatives are one of the most promising compounds on anti-inflammatory drug formulation.” (Salehi, 2018). “The so-called ‘Resveratrol Paradox’, i.e., low bioavailability but high bioactivity, is a conundrum not yet solved in which the final responsible actor (if any) for the exerted effects has not yet been unequivocally identified. It is becoming evident that resveratrol exerts cardioprotective benefits through the improvement of inflammatory markers, atherogenic profile, glucose metabolism and endothelial function. However, safety concerns remain unsolved regarding chronic consumption of high RES doses, especially in medicated people. This review will focus on the currently available evidence regarding resveratrol's effects on humans obtained from randomized clinical trials. In addition, we will provide a critical outlook for further research on this molecule that is evolving from a minor dietary compound to a possible multi-target therapeutic drug.” (Tomé-Carneiro, 2013).

### Scientific Articles:

a. Salehi, B. et al. 2018. Review, Resveratrol: A Double-Edged Sword in Health Benefits. Biomedicines. 6: 91-110.
b. Tome-Carneiro, J. et al. 2013. Resveratrol and Clinical Trials: The Crossroad From In vitro Studies to Human Evidence. Curr Pharm Des. 19(34):6064-93.

### 13. Boron Citrate

“The trace mineral boron is a micronutrient with diverse and vitally important roles in metabolism that render it necessary for plant, animal, and human health, and as recent research suggests, possibly for the evolution of life on Earth” (Pizzorno, 2015).

Industry Claimed Uses: strong bones, wound healing, anti-inflammatory, building muscles, improved thinking.

### Science Related Genetic Application:

Scientific papers have indicated that boron has important anti-inflammatory activity (Naghii, 2011). With respect to inflammatory biomarkers, this mineral reduces levels of CRP and TNF-α; raises levels of antioxidant enzymes, such as SOD, catalase, and glutathione peroxidase. In addition, boron protects against pesticide-induced oxidative stress and heavy-metal toxicity (Pizzorno, 2015).
Another important attribute is the finding that boron increases the biological half-life and bioavailability of the major female hormone, estradiol, and vitamin D (Miljkovic, 2004).

### Scientific Articles:

a. Miljkovic, D. 2004. Up-regulatory impact of boron on vitamin D function-does it reflect inhibition of 24-hydroxylase? Med Hypotheses. 63(6):1054-1056.
b. Naghii M R. et al. 2011. Comparative effects of daily and weekly boron supplementation on plasma steroid hormones and proinflammatory cytokines. J Trace Elem Med Biol. 25(1):54-58.
c. Pizzorno, L. 2015. Nothing Boring About Boron. Integr Med (Encinitas). 14(4): 35-48.

### 14. Magnesium Diglycinate

Magnesium is an essential dietary mineral. It is the second most common deficiency in developed countries; The first being vitamin D. A deficiency increases blood pressure, reduces glucose tolerance and causes neural excitation. If magnesium is supplemented, it acts as a sedative, reducing blood pressure and improving insulin sensitivity (Schwalfenberg, 2017).

Industry Claimed Uses: Protection against depression and ADHD, reduction of muscle cramps, better sleep, strong bones.

### Science Related Genetic Application:

In a study by the U.S. Department of Agriculture, “magnesium citrate supplementation compared to a sodium citrate placebo decreased plasma CRP in participants with values above 3.0 mg/dL indicates that subclinical magnesium deficiency may exacerbate conditions that result in chronic inflammatory stress (Nielsen, 2010).
A non-human study showed that “Mg deficiency, independently of any other changes in nutrient intake, modulates the concentration of bifidobacteria in the gut, a phenomenon that may time-dependently affect inflammation and metabolic disorders in mice (Pachikian, 2010).

### Scientific Articles:

a. Schwalfenberg, G K and Genuis, S J. 2017. Scientifica Volume. Article ID 4179326, 14 pages.
b. Nielsen, F H. et al. 2010. Magnesium supplementation improves indicators of low magnesium status and inflammatory stress in adults older than 51 years with poor quality sleep. Magnesium Research. 23 (4): 158-68.
c. Pachikian, Bd, et al. 2010. Changes in Intestinal Bifidobacteria Levels Are Associated with the Inflammatory Response in Magnesium-Deficient Mice. The Journal of Nutrition. 140 (3),509-514.

### 15. Curcumin

Curcumin is the main active ingredient in a spice called turmeric which comes from the Curcuma longa plant, native to Asia and Central America. Curcumin is a natural anti-inflammatory compound which has the potential to dramatically increase the antioxidant capacity of the body.

Industry Claimed Uses: Improved brain function, muscle soreness, exercise-induced inflammation.

### Science Related Genetic Application:

Research suggests that curcumin can help in the management of oxidative and inflammatory conditions, metabolic syndrome, arthritis, anxiety, and hyperlipidemia (Hewlings, 2017).
Tumor necrosis factor α (TNF-α), a multifunctional cytokine, is a major mediator of inflammation in most diseases and this effect is regulated by the activation of a transcription factor, nuclear factor (NF)-κB. In addition to TNF-α, NF-κB is also activated by most inflammatory cytokines; gram-negative bacteria; various disease-causing viruses; environmental pollutants; chemical, physical, mechanical, and psychological stress; high glucose; fatty acids; ultraviolet radiation; cigarette smoke; and other disease-causing factors. Therefore, agents that downregulate NF-κB and NF-κB-regulated gene products have potential efficacy against several of these diseases. Curcumin has been shown to block NF-κB activation increased by several different inflammatory stimuli [Panahi, 2016).
Studies have also revealed the potential for curcumin to reduce circulating C-reactive protein (CRP) levels in the blood (Panahi, 2015).

### Scientific Articles:

a. Hewlings, S J and Kalman, D S. 2017. Curcumin: A Review of Its' Effects
b. on Human Health. Foods. 6: 92.
c. Panahi, Y. et al. 2016. Effects of curcumin on serum cytokine concentrations in subjects with metabolic syndrome: A post-hoc analysis of a randomized controlled trial. Biomed. Pharmacother. 82: 578-582.
d. Panahi, Y. et al. 2015 Antioxidant and anti-inflammatory effects of curcuminoid-piperine combination in subjects with metabolic syndrome: A randomized controlled trial and an updated meta-analysis. Clin. Nutr. 34: 1101-1108.

### 16. Folic Acid (L-Methylfolate)

Folate and folic acid are different forms of vitamin B9. The natural form is folate and its name is derived from the Latin word “folium,” which means leaf. In fact, leafy vegetables are among the best dietary sources of folate. The active form of folate is essential for the body to make DNA and RNA and metabolize amino acids, which are required for cell division. As humans cannot make folate, it is required in the diet, making it an essential vitamin. Approximately 85% of people with serious kidney disease have high levels of homocysteine which has been linked to heart disease and stroke. Taking folic acid lowers homocysteine levels (Modaghegh, 2016).

Industry Claimed Uses: Prenatal health (Junod,2001), strong blood, lowers homocysteine levels, reduces risk of eye disease, memory, depression.

### Science Related Genetic Application:

A clinical research trial was performed, in Tianjin, China, to evaluate whether folic acid supplementation would improve cognitive (memory) performance by reducing inflammatory cytokine concentrations. Neuropsychological tests were administered, and folate, homocysteine, vitamin B12, IL-6, TNF-α, Δβ-42, and Δβ-40 were measured at baseline and at 6- and 12-month time points. 152 participants (folic acid: 77, conventional: 75) completed the trial. Significant improvements in folate, homocysteine, Δβ-42, peripheral IL-6, TNF-α levels were observed in folic acid group compared with conventional group. Full scale intelligence quotient also improved in the folic acid group (Ma, 2016).
A randomized trial at Brigham and Women's Hospital in a large cohort of women at high risk of cardiovascular disease indicate that daily supplementation with folic acid/B6/B12 may reduce the risk of age-related macular degeneration (AMD) (Christen, 2009).

### Scientific Articles:

a. Christen, W G, et al. 2009. Folic Acid, Vitamin B6, and Vitamin B12 in Combination and Age-related Macular Degeneration in a Randomized Trial of Women. Arch Intern Med. 2009 February 23; 169(4): 335-341.
b. Junod, S W. 2001. Folic Acid Fortification: Fact or Folly. Update, the bimonthly publication of the Food and Drug Law Institute. www.fda.gov
c. Ma, F, et al. 2016. Folic acid supplementation improves cognitive function by reducing the levels of peripheral inflammatory cytokines in elderly Chinese subjects with MCI. Scientific Reports. 6:
d. Article number: 37486.
e. Modaghegh, M H S, et al. 2016. Effect of Folic Acid therapy on Homocysteine Level in patients with Atherosclerosis or Buerger's Disease and in Healthy individuals: A clinical trial. Electronic Physician. 8 (10): 3138-3143.

### 17. Niacin

Niacin is one of the eight B vitamins, and it is also called vitamin B3. It is water soluble and is not stored by the body. Excess amounts can therefore be excreted, if not needed. The body keeps a small reserve of this vitamin, but it must be taken on a regular basis to maintain the reserve.

Industry Claimed Uses: Memory loss and mental confusion, depression, fatigue, skin problems, headache.

### Science Related Genetic Application:

In vivo study revealed that niacin down-regulated the levels of inflammatory factors (IL-6 and TNF-α) in plasma, suppressed protein expression of CD68 and NF-κB p65 in the arterial wall, and attenuated oxidative stress in guinea pigs that have been fed high-fat diet (Si, 2014).
Niacin Decreased the Secretion of Inflammatory Cytokines TNF-α and IL-6 in oxLDL (oxidized low-density lipoproteins)-Stimulated HUVECs (Human umbilical vein endothelial cells) (Pirillo, 2013).
Oxidized LDL (OxLDL) activates endothelial cells in blood vessel and is part of the process involved formation of an atheromatous plaque. Atherosclerosis is a chronic inflammatory vascular disease, having as ultimate outcome the atheromatous plaque, a focal lesion located within the intima of large and medium sized arteries (Libby, 2012).
Niacin significantly lessened lipid deposition in the arterial wall and modified lipoprotein profile in plasma via modulating cholesterol metabolism in liver of guinea pigs fed high fat diet. (Si, 2014).
“In summary, our data presented herein support the novel concept that niacin has vascular anti-inflammatory and potentially vascular-protective property which is independent of its effect on lipid regulation. The anti-inflammatory property of niacin is realized by downregulating the nuclear transcription factor-KB signaling pathway.” (Si, 2014).

### Scientific Articles:

a. Libby, P. 2012. Inflammation in atherosclerosis. Arteriosclerosis, Thrombosis, and Vascular Biology. 32(9):2045-2051.
b. Pirillo, A, et al. 2013. LOX-1, OxLDL, and Atherosclerosis. Mediators of Inflammation. 2013:12 pages. 152786 [PubMed].
c. Si, Y, et al. 2014. Niacin Inhibits Vascular Inflammation via Downregulating Nuclear Transcription Factor-KB Signaling Pathway. Hindawi Publishing Corporation Mediators of Inflammation Volume 2014, Article ID 263786, 12 pages.

### 18. Riboflavin

A water-soluble vitamin, also known as vitamin B2. It is required by the body for cellular respiration. It is on the World Health Organization's List of Essential Medicines, the safest and most effective medicines needed in a health system (WHO, 2019). It is continuously excreted in the urine of healthy individuals, making deficiency relatively common when dietary intake is insufficient (Brody, 1999).

Industry Claimed Uses: Riboflavin deficiency, growth, good health, decrease cataract risk, migraines, decrease homocysteine levels.

Science Related Genetic Application: Riboflavin is claimed to have anti-inflammatory, antioxidant, and microbiome-modulatory properties. A Crohn's disease (CD) study revealed that three weeks of riboflavin supplementation resulted in a reduction in systemic oxidative stress, mixed anti-inflammatory effects, and a reduction in clinical symptoms. IL-2 and C-reactive protein (CRP) levels were reduced in certain patient groups. “Our data demonstrate that riboflavin supplementation has a number of anti-inflammatory and anti-oxidant effects in CD.” (von Martels, 2020).

“Riboflavin by its proteasome inhibitory action down regulates the NF-up pathway, thus reducing pro-inflammatory cytokines, nitric oxide and COX2, which ensures protection from infection on one hand and survival benefit of host cells from inflammatory damage on the other. The balance between the redox reactions and antioxidant system suggests a strong therapeutic intervention in serious bacterial infections associated with sepsis at least in our in-vitro system [FIG. 9].” (Dey, 2016).

### Scientific Articles:

a. Brody, T. 1999. Nutritional Biochemistry. San Diego: Academic Press.
b. Dey, S. and Bishayi, B. 2016. Riboflavin along with antibiotics balances reactive oxygen species and inflammatory cytokines and controls Staphylococcus aureus infection by boosting murine macrophage function and regulates inflammation. Journal of Inflammation. 13:36.
c. Von Martels, J Z H, et al. 2020. Riboflavin Supplementation in Patients with Crohn's Disease [The RISE-UP Study]. J Crohns Colitis. 14(5):595-607.
d. World Health Organization (2019). World Health Organization model list of essential medicines: 21st list 2019. Geneva: World Health Organization.

### 19. Thiamin

Thiamin, vitamin B1, is an essential vitamin that has many important functions in the body. It is used by most cells to help convert food into energy. Many people don't realize that they have a deficiency, as many of the symptoms are subtle and often overlooked. Risk of deficiency is increased by alcohol dependence, old age, diabetes, dialysis, HIV/AIDS, and high-dose diuretic use. Severe thiamin deficiency leads to beriberi, a disease that affects multiple organ systems, including the central and peripheral nervous systems. Beriberi was described in Chinese literature as early as 2600 B.C.

Industry Claimed Uses: Aids in boosting the immune system, diabetic pain, heart disease, heart failure, alcoholism, aging, cataracts and glaucoma.

Science Related Genetic Application: Experiments feeding thiamin deficient chow to septic animals showed an increase in levels of TNF-α in their peritoneal fluid. TNF-α is an early marker of inflammation commonly used in infection models, such as sepsis. TD (sic. thiamine deficiency) was associated with a greater bacterial clearance in the peritoneal fluid, greater oxidative stress and a change in the immune response profile in an experimental model of abdominal sepsis in mice (Andrade, 2014).

### Scientific Articles:

a. De Andrade, J A A, et al. 2014. The effect of thiamine deficiency on inflammation, oxidative stress and cellular migration in an experimental model of sepsis. Journal of Inflammation. 11:11.

### 20. Selenium (SE)

An essential mineral that is a component of an antioxidant enzyme, glutathione reductase, that is key in tissue respiration. Selenium is a powerful mineral that is essential for the proper functioning of your body. It is only needed in small amounts but plays a critical role in metabolism and thyroid function and helps protect your body from damage caused by oxidative stress. Antioxidants like selenium reduce oxidative stress by neutralizing and keeping free radical numbers in check.

Industry Claimed Uses: Antioxidant, anti-inflammatory, cellular protection, Crohn's disease.

Science Related Genetic Application: “Even though the pathophysiology of irritable bowel disease (IBD) is multifactorial in origin, dietary selenium (and selenoprotein) deficiency exacerbates experimental colitis by affecting various signaling pathways involved in inflammation and oxidative stress as well as by altering the gut microbiota.” (Kudva, 2015).

The glutathione peroxidase (GPX) enzymes utilize Se at their active sites to detoxify reactive oxygen species (ROS) including hydrogen peroxide (H2O2) and phospholipid hydroperoxide. GPX1 and 4 are among the most abundant selenoproteins in several immune cells and tissues (Carlson, 2010). Selenium, in a mouse model of diabetes, appears to have a potential protective effect in type 1 diabetes. This protective effect involved a shift in the balance between inflammatory cytokines (TNF-α) and regulatory cytokines (IL-10) (Huang,2012)

### Scientific Articles:

a. Carlson, B A, et al. Role of selenium-containing proteins in T-cell and macrophage function. Proc Nutr Soc. 69:300-310.
b. Huang, Z, et al. 2012. The Role of Selenium in Inflammation and Immunity: From Molecular Mechanisms to Therapeutic Opportunities. Antioxid Redox Signal. 16(7): 705-743.
c. Kudva, A K, et al. 2015. Selenium and inflammatory bowel disease. Am J Physiol Gastrointest Liver Physiol. 309(2): G71-G77.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Leveraging the Unique Language of Cellular Communication

Cellular communication is a dynamic system that adjusts itself in response to the local and systemic cellular environment. For example, the immune system uses local and systemic signal transduction mechanisms to defend the body from disease or invading pathogens. Normally, the immune response is activated and then promptly terminated to avoid excessive tissue damage. In an inflammatory condition such as Covid pulmonary cytokine storm the immune response is sustained and out of control by the body and excessive lung damage occurs.

### Example 2: Immune Response and the Production of Inflammatory Cytokine Signals is the Response of Brain Tissue to a Bacterial Blood Infection, Endotoxemia

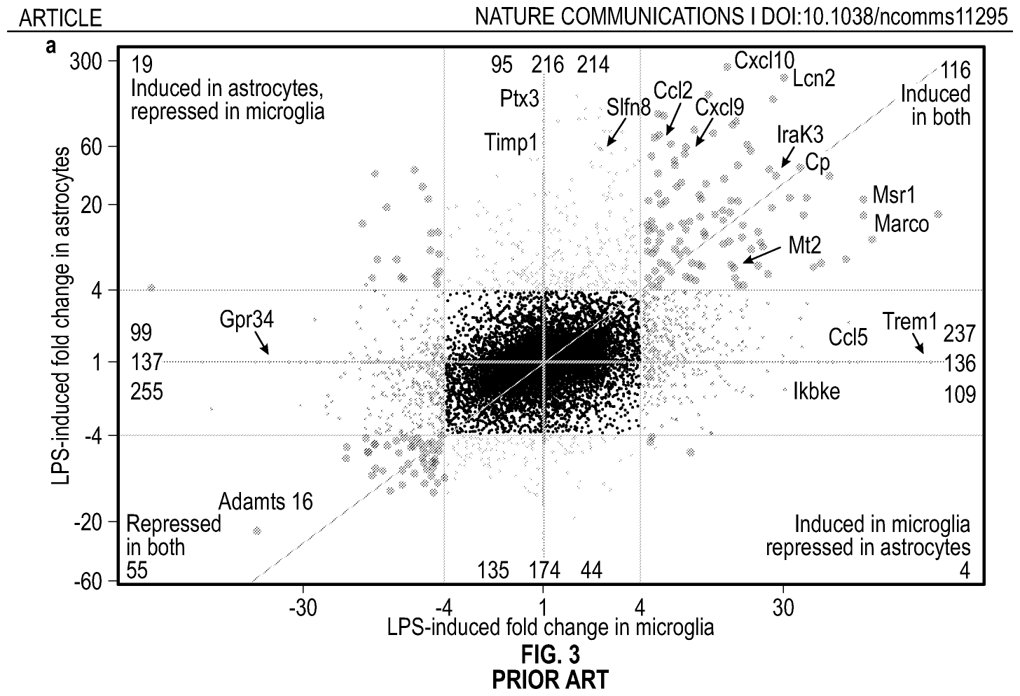

FIG. 3 shows the inflammatory genetic response within the brain of a mouse to a bacterial blood infection. The various signals show different gene expression profiles for the immune cells in the brain which mediate many aspects of the inflammatory response. The peripheral endotoxemia had a profound effect on gene expression in both microglia and astrocytes, as samples within these cell types clustered naturally into treatment and control groups (Srinivasan, 2016).

### Example 3: Leveraging Differential Expression of mRNA for Cancer Diagnostics Background

Messenger RNA (mRNA) represents an emerging genomic science that has led to the development of vaccines against coronavirus and the treatment of cancer. mRNA can also be used to evaluate the expression of inflammatory proteins, called cytokines and chemokines, that are involved in immune system regulation, tissue damage and repair, genetic disorders, and it can also be applied to immunotherapy through the expression of antigens, antibodies or receptors.

mRNA is the only coding RNA in organisms and is the ‘middleman’ of gene expression. Genetic information in DNA is communicated by mRNA to protein manufacture machinery, along with regulatory systems for protein expression and translation.

In cancer evaluation and treatment, mRNA can be used as a biomarker and a target for cancer therapy, and the mRNA responsible for encoding tumor antibodies also has the potential to evoke effective anti-tumor immunity (Obeng E A, et al. 2019. Altered RNA Processing in Cancer Pathogenesis and Therapy. Cancer Discov. 9(11):1493-510). At present, mRNA plays an increasingly important role in the occurrence and development of tumors, treatment and diagnosis, and in recent years, mRNA tumor vaccines have been gradually applied to cancer treatment; in addition, mRNA therapy has also been beginning to play a role in the treatment of various types of tumors as an emerging gene therapy (Coelho M A, et al. 2017. Oncogenic RAS Signaling Promotes Tumor Immunoresistance by Stabilizing PD-L1 mRNA. Immunity.47(6):1083-1099; Zheng Y, et al. 2021. The role of mRNA in the development, diagnosis, treatment and prognosis of neural tumors. Molecular Cancer. 20, Article number: 49). Although cancer cells produce a variety of chemokine ligands that may be involved in neoangiogenesis, attraction and retention of inflammatory cells, and tumor cell proliferation, neoplastic cells express a limited repertoire of chemokine receptors. These receptors play distinct roles in distant organ metastasis (Muller A, et al. 2001. Involvement of chemokine receptors in breast cancer metastasis. Nature. 410:50-56; Murakami T et al. 2003. Immune evasion by murine melanoma mediated through CC chemokine receptor-10. J Exp Med 198:1337-1347).

Three criteria are required for the immunologic destruction of established tumors: (i) sufficient numbers of immune cells with highly avid recognition of tumor antigens must be generated in vivo (ii) these cells must traffic to and infiltrate the tumor stroma, and (iii) the immune cells must be activated at the tumor site to manifest appropriate effector mechanisms such as direct lysis or cytokine secretion capable of causing tumor destruction (Rosenberg S, et al. 2004. Cancer immunotherapy: Moving beyond current vaccines. Nat Med. 10:909-915).

### Current Regulatory Landscape for Biomarkers

The fact that development of better evaluation tools and biomarkers was named a top priority of the FDA's Critical Path Opportunities Report in 2006 underlined the importance of defining biomarkers validation and qualification processes (FDA. Critical path opportunities report. 2006, at www.slcmsr.net/download/flyer/Critical_Path_Initiative Report_FDA_2006.pdf). On average around 65% of drug approvals by EMA and FDA between 2015 and 2019 have been associated with incorporation of at least 1 biomarker in the development program and higher percentage of biomarker acceptance is expected in the near future (Gromova M, et al. 2020. Biomarkers: Opportunities and Challenges for Drug Development in the Current Regulatory Landscape. Biomarker Insights. 15: 1-15).

mRNA Biomarkers

mRNA transcripts are becoming very important as molecular biomarkers for the diagnosis and treatment of many diseases, cancer being very important. The mRNA transcript carries the gene's protein information encoded in DNA. The mRNA moves from the cell nucleus to the cell cytoplasm where it is used for synthesizing the encoded protein. These biomarkers offer early and more accurate prediction and diagnosis of disease and disease progression, and the ability to identify individuals at risk. “Research is needed to develop mRNA biomarker panels for all nutrients that will discriminate between deficient, marginal, adequate, and supernutritional individuals and populations, and differentiate between individuals that will benefit versus be adversely affected by nutrient supplementation.” (Sunde R A. 2010. mRNA transcripts as molecular biomarkers in medicine and nutrition. J Nutr Biochem. 21(8): 665-670) We describe a method to use buccal or cheek cells as a surrogate tissue from which to isolate sufficient nanogram amounts of mRNA that can be measured in microarray chips.

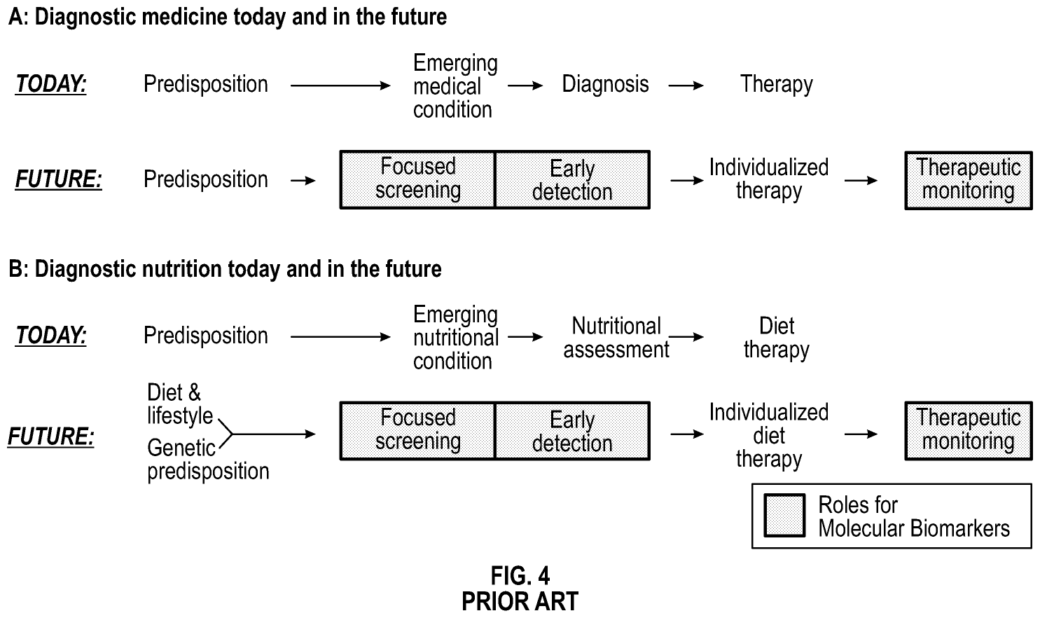

In his essay “Predicting disease using genomics,” Bell suggests that genetic (genes) and genomic (RNA and protein) information will allow early and more accurate prediction and diagnosis of disease and disease progression, and that medicine will become oriented towards disease prevention rather than efforts to cure people at late stages of illness (Bell J. 2004. Predicting disease using genomics. Nature. 429:453-6). Bell foresees that screening via molecular biomarkers for genetic predisposition will facilitate early detection, define individual disease subtypes based precisely on individual pathophysiology, and will inform treatment and responsiveness to therapy (FIG. 4). FIG. 4 shows the impact of molecular biomarkers in medicine and in nutrition, leading to individualized therapy. (A) Diagram of diagnostic medicine today and in the future (adapted from Bell, Id. by permission). (B) Diagram of diagnostic nutrition today and in the future. Boxes show key potential roles for molecular biomarkers to provide early detection of aberrant nutritional status due to genetics or diet/lifestyle, thus shaping individualized therapy and subsequent monitoring.

It is well accepted scientifically that cytokines and chemokines play an important role in cancer development and control. Cytokines affect the growth of all blood cells and other cells that help the body's immune and inflammation responses. They also help to boost anti-cancer activity by sending signals that can help make abnormal cells die and normal cells live longer. One specific type of cytokine is called a chemokine. The findings presented in this patent are based on the role that mRNAs are involved in the post-transcriptional regulation of cytokine expression.

Our final chip quantified expression levels of the following 48 mRNA's: ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, IGF1, IL-1(IL1A), IL-10, IL-18, IL-1B, IL-6, KRT1, KRT83, MMP13, MUC1, NFKB1, NFKB2, NLRP3, NLRP1, NR3C1, PAX8, PDL1(CD274), PPARG, RGS2, SDK1, SIRT1, SKA2, SLC11A2, SLC22A4, SLC40A1, SLC6A3, SMAD3, STK11,TGFB1, TNF, VEGFA.

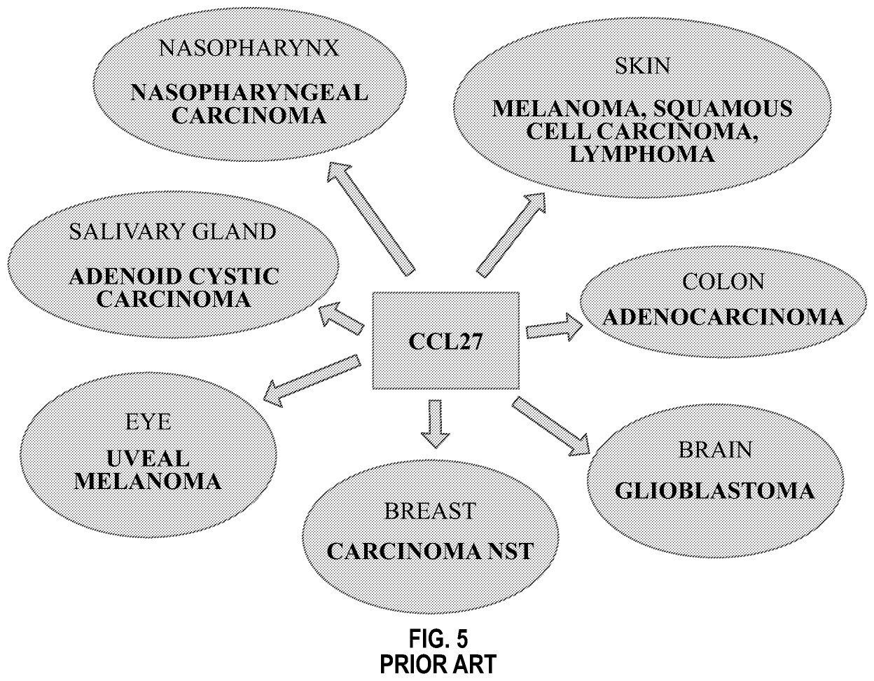

To Illustrate, CCL27 is a scientifically well-accepted chemokine that is overexpressed in certain types of squamous cell carcinoma and expressed at various levels in metastatic prostate, breast, colorectal, and pancreatic cancer as well as melanoma cell lines (Karnezis T et al 0.2019. CCL27/CCL28-CCR10 chemokine signaling mediates migration of lymphatic endothelial cells. Cancer Res 79:1558-1572) (FIG. 5).

FIG. 5 illustrates the major human tissues in which CCL27 plays an important role in tumor progression and the main types of tumors which are influenced by CCL27 (from Martinez-Rodriguez M and Monteagudo C. 2021. CCL27 Signaling in the Tumor Microenvironment. Tumor Microenvironment, Advances in Experimental Medicine and Biology 1302).

Chemokines such as CCL27 play an important role in mediating the recruitment of the appropriate immune cells to the tumor and establishing effective antitumor immunity. The key to this process is to recognize malignant cells which are eliminated by tumor-associated antigen-specific T cells, thereby preventing tumor progression (Pawelec, G. 2004.Immunotherapy and immunoselection—tumor escape as the final hurdle. FEBS Lett 567:63-66).

### Materials and Methods

### Observational Protocol

Accumulate quantitative mRNA data that will undergo genetic analyses leveraging machine learning-derived embedding and clustering to identify markers of genetic expression involved in cancer.

### Study Population and Recruitment

Patients with personal cancer history were admitted into the study. Various cancer types accepted include but are not limited to breast cancer, colon cancer, bladder cancer, intestinal cancers, gynecologic cancer, skin cancer, lung cancer, and lymphatic system cancers. Physician consultation and/or genetic consultation were obtained for each individual enrollee, as needed.

### Genetic Testing

Genomic RNA was extracted from a buccal swab sample using a standard kit. The individual to be tested swabbed the right and left cheeks with a cotton swab and placed the swabs in envelopes or tubes labeled specifically for RNA analysis. We created a custom chip for reading candidate mRNA-based cytokine biomarkers, selected, based on association with inflammatory pathways and other systems of interest in development of cancers. Our final chip quantified expression levels of the following 48 mRNA's: ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, IGF1, IL-1(IL1A), IL-10, IL-18, IL-1B, IL-6, KRT1, KRT83, MMP13, MUC1, NFKB1, NFKB2, NLRP3, NLRP1, NR3C1, PAX8, PDL1(CD274), PPARG, RGS2, SDK1, SIRT1, SKA2, SLC11A2, SLC22A4, SLC40A1, SLC6A3, SMAD3, STK11, TGFB1, TNF, VEGFA.

### Wet Lab Methods Description

Sample collection was carried out with swabs immediately placed in a screw cap tube containing 0.5 ml of molecular collection and preservation medium plus one regular flocked nylon fiber swab following collection. This type of swab stabilizes and preserves nucleic acid for prolonged periods of time. Sample integrity was maintained at ambient temperature for transportation via regular post. The transport media and stabilizing liquid helped to keep the sample protected, minimizing the growth of bacteria until processing can take place. Samples were stored at a low temperature in the lab.

Extraction was performed using a compact, automated nucleic acid purification platform that processes up to 48 samples simultaneously. This procedure uses paramagnetic particles to provide a mobile solid phase to optimize sample capture, washing and purification of nucleic acid. As soon as the run was complete, samples were extracted and placed on ice.

Samples were quantified using a benchtop fluorometer to measure the RNA concentration (ng/uL). Following the quantification, the RNA sample concentration was normalized. We used multiplexing to help maximize the use of the ever-increasing capacity of NGS technology, allowing us to run multiple libraries while minimizing costs.

Before starting the library, RNA samples were subjected to a process via reverse transcription that produces complementary DNA (cDNA). For this process the reverse transcriptase (RTs) uses an RNA template and a short primer complementary to the 3′ end of the RNA to direct the synthesis of the first strand cDNA.

The construction of sequencing libraries started with the cDNA target amplification, where the specific panel designed for this study was incorporated into the samples and amplified. A partial digest of the amplicons was carried out, generating the specific amplicons needed. Ligation reaction included the addition of the unique adapters or barcodes to each of the samples, followed by a purification of the library, where all the excess of reagents added were removed. Following this, we normalized all libraries included in the same batch, in this way during the sequencing process all the libraries were treated equally, as they will be present in similar concentrations. After a final cleanup in which all the excess components not used were removed, the libraries were ready for the last final step, which included polling libraries followed by the sequencing.

The resulting library was sequenced on a commercially available next-generation sequencer using commercially available reagents and kits. Output files were transferred to a bioinformatics pipeline for further analysis.

### Results and Analysis

Data analysis was performed using Applied Biosystems' Transcriptome Analysis Console (TAC) Software, from which we obtained a list of differentially expressed biomarkers. P-values and conditional false discovery rate-corrected p-values (FDR) from F-tests were used to filter genes, where FDR is defined as the experiment-wise threshold for statistical significance. We report biomarkers as individually differentiable for the cancers in question when the respective FDR p-values are below the threshold of α=0.05; biomarkers achieving a p-value less than a without FDR correction were also considered significant.

Subjects were excluded from the differential biomarker analysis if their cancer was labeled “metastatic”, in an effort to decrease potential sources of noise. Following this, one additional breast cancer subject was removed because their quantified expression levels significantly differed from all other subjects.

Tables 3-5 show our results comparing biomarker expression in various cancers. Importantly, all average log 2 expression levels reported here are rounded to the hundredths place, so “0” does not necessarily indicate a true absence of expression levels. In Table 3, we see differential expression of KRT1 and CCL27 in breast and colon cancer using FDR p-values; SDK1 and PPARG showed differential expression using p-values without FDR correction. Table 4 compares biomarker expression in bladder and colon cancer. Here, we see IL1A and SMAD3 showed differential expression using p-values without FDR correction, IL1A nearly achieves FDR-corrected significance. Table 5 depicts results for differential expression of bladder and colon cancer. IL1A and CCL27 show differential expression using p-values without FDR correction.

Table 6 reports the results of our one-way ANOVA examining the relationship of cancer diagnosis to biomarker levels. Both FDR-corrected and uncorrected p-values showing significance are reported. SLC6A3, NR3C1, KRT1, KRT83, HFE2, CCL27, ILIA, SMAD3, CASP3, SDK1, CACNA1C, NFKB2, PPARG, CASP9 showed differential expression using p-values without FDR correction; SLC6A3, NR3C1, KRT1, KRT83, HFE2, CCL27 showed differential expression using p-values with FDR correction.

TABLE 3
Differential expression of individual
biomarkers for breast vs. colon cancer

| Gene | Breast | Colon | Fold | | FDR |
|---|---|---|---|---|---|
| Symbol | Avg (log2) | Avg (log2) | Change | p-value | p-value |
| KRT1 | 14.22 | 15.96 | −3.34 | 0.0004 | 0.0063 |
| CCL27 | 0 | 1.23 | −2.34 | 0.0023 | 0.0188 |
| SDK1 | 0 | 1.76 | −3.38 | 0.0131 | 0.0694 |
| PPARG | 8.03 | 9.14 | −2.17 | 0.0159 | 0.0694 |

TABLE 4
Differential expression of individual
biomarkers for bladder vs. colon cancer

| Gene | Bladder | Colon | Fold | | FDR |
|---|---|---|---|---|---|
| Symbol | Avg (log2) | Avg (log2) | Change | p-value | p-value |
| IL1A | 0 | 4.22 | −18.61 | 0.0024 | 0.0585 |
| SMAD3 | 10.92 | 12 | −2.12 | 0.0094 | 0.0937 |

TABLE 5
Differential expression of individual biomarkers
for bladder vs. breast cancer

| Gene | Bladder | Breast | Fold | | FDR |
|---|---|---|---|---|---|
| Symbol | Avg (log2) | Avg (log2) | Change | p-value | p-value |
| IL1A | 0 | 3.1 | −8.58 | 0.0094 | 0.3766 |
| CCL27 | 1.75 | 0 | 3.37 | 0.0297 | 0.3766 |

TABLE 6
Results of one-way ANOVA comparing cancer
diagnosis with biomarker expression levels

| | Bladder | Breast | Colon | | |
|---|---|---|---|---|---|
| Gene | Avg | Avg | Avg | | FDR |
| Symbol | (log2) | (log2) | (log2) | p-value | p-value |
| SLC6A3 | 0 | 0 | 0 | 0.0003 | 0.0097 |
| NR3C1 | 13.23 | 13.66 | 13.02 | 0.0004 | 0.0097 |
| KRT1 | 15.35 | 14.22 | 15.96 | 0.0024 | 0.0379 |
| KRT83 | 0 | 0 | 0 | 0.0032 | 0.0379 |
| HFE2 | 0 | 0 | 0 | 0.0056 | 0.0453 |
| CCL27 | 1.75 | 0 | 1.23 | 0.0057 | 0.0453 |
| IL1A | 0 | 3.1 | 4.22 | 0.0075 | 0.0513 |
| SMAD3 | 10.92 | 11.28 | 12 | 0.022 | 0.1322 |
| CASP3 | 12.85 | 13.46 | 13.64 | 0.0264 | 0.141 |
| SDK1 | 0 | 0 | 1.76 | 0.0367 | 0.1705 |
| CACNA1C | 0 | 0 | 0 | 0.0442 | 0.1705 |
| NFKB2 | 14.01 | 13.9 | 14.34 | 0.0446 | 0.1705 |
| PPARG | 7.5 | 8.03 | 9.14 | 0.0487 | 0.1705 |
| CASP9 | 12.14 | 11.96 | 12.56 | 0.0497 | 0.1705 |

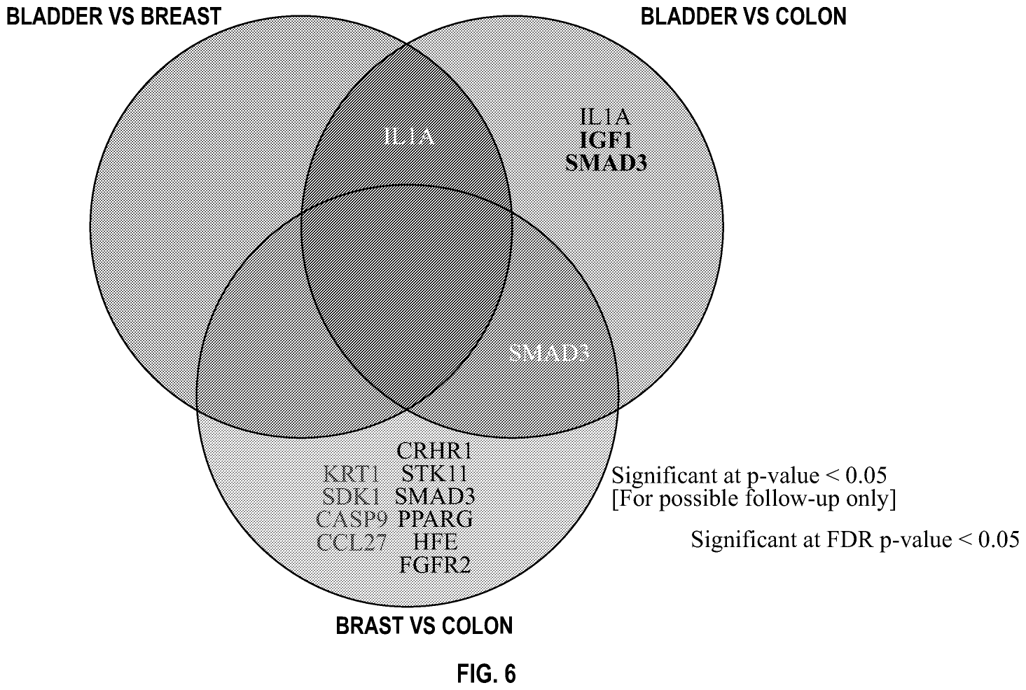

These results are summarized in FIG. 6, a Venn diagram showing overlap of the various genes showing significance.

### CONCLUSION

Due to the different types of cancers and the frequent emergence of cancer symptoms at advanced stages, it is challenging to detect cancer at an early stage. Identifying broad and reliable biomarkers for cancer detection and prediction in the early stage may provide timely treatment for patients. The apportionment of complex diseases, such as cancer, into simpler sub-classifications through the use of novel biomarkers and signature profiles will improve patient medical care, outcomes, as well as our mechanistic understanding of cancer. Diseases that are more similar mechanistically will be separated into distinct categories earlier in their pathophysiological progression. Molecular phenotyping using mRNA genetic and genomic information will allow early and more accurate prediction and diagnosis of disease and of disease progression. Disease prevention rather than efforts to cure people at late stages of illness will be the result.

Genomic and proteomic approaches to cancer evaluation and treatment have the potential to revolutionize our understanding of disease initiation and progression. Specifically, we can classify illness on a molecular basis for better diagnostic and prognostic precision, and design personalized therapies tailored to the individual.

A key advantage offered by a systems biology strategy is that it permits an unbiased characterization of the genes and proteins of interest, thereby allowing a characterization of gene and protein interactions in a systematic fashion. By combining this approach with knowledge of gene variants, we can optimize our capacity for identifying key targets in disease pathways.

In this study, we describe the application of differentially expressed mRNA biomarkers obtained via non-invasive buccal swab to distinguish between types of cancers.

### REFERENCES

Abdulkhaleq, L. A., Assi, M. A., Abdullah, R., Zamri-Saad, M., Taufiq-Yap, Y. H., & Hezmee, M. (2018). The crucial roles of inflammatory mediators in inflammation: A review. Veterinary world, 11(5), 627-635. https://doi.org/10.14202/vetworld.2018.627-635
Advanced Center for Pain and Rehab, 2017, Exposing America's Silent Killer: Chronic inflammation.
Alexander M, et al. 2015. Exosome-delivered microRNAs modulate the inflammatory response to endotoxin. Nat Commun. 6.7321.
Amin M N, Siddiqui S A, Ibrahim M, Hakim M L, Ahammed M S, Kabir A, Sultana F. Inflammatory cytokines in the pathogenesis of cardiovascular disease and cancer. SAGE Open Med. 2020 Oct. 20; 8:2050312120965752. doi: 10.1177/2050312120965752. PMID: 33194199; PMCID: PMC7594225.
Anyanwu,AC.,Kanthi,Y.et al.2019.TuningtheThrombo-inflammatory Response to Venous Flow Interruption by the Ectonucleotidase CD39. Arteriosclerosis, Thrombosis, and Vascular Biology. Volume 39, Issue 4, Pages el 18-e129.
Beckett E L, et al. 2014. The Role of Vitamins and Minerals in Modulating the Expression of microRNA. Nutr Res Rev. 27(1):94-106.
BRUNO RICARDO BARRETO PIRES, iSIS SALVIANO SOARES DE AMORIM, LAYANE DUARTE E SOUZA, JULIANA ALVES RODRIGUES, ANDRE LUIZ MENCALHA. Targeting Cellular Signaling Pathways in Breast Cancer Stem Cells and its Implication for Cancer Treatment. Anticancer Research Nov 2016, 36 (11) 5681-5691;
Byun, J S and Gardner, K. 2013. Wounds That Will Not Heal, Pervasive Cellular Reprogramming in Cancer. Am. J. of Path. 182 (4): 1055-1064.
Carlos Salomon, Dominic Guanzon, Katherin Scholz-Romero, Sherri Longo, Paula Correa, Sebastian E Illanes, Gregory E Rice, Placental Exosomes as Early Biomarker of Preeclampsia: Potential Role of Exosomal MicroRNAs Across Gestation, The Journal of Clinical Endocrinology & Metabolism, Volume 102, Issue 9, 1 Sep. 2017, Pages 3182-3194, https://doi.org/10.12101c.2017-00672
Closa, D. & Folch-Puy, E. 2004. Oxygen Free Radicals and the Systemic Inflammatory Response. Life, 56(4): 185-191.
Cornelius D. C. (2018). Preeclampsia: From Inflammation to Immunoregulation. Clinical medicine insights. Blood disorders, 11, 1179545X17752325. https://doi org/10.1177/1179545X17752325
Dowsett, J., Didriksen, M., von Stemann, J. H. et al. Chronic inflammation markers and cytokine-specific autoantibodies in Danish blood donors with restless legs syndrome. Sci Rep 12, 1672 (2022). https://doi.org/10.1038/s41598-022-05658-1
Esquivel-Velizquez M, Ostoa-Saloma P, Palacios-Arreola M I, Nava-Castro K E, Castro J I, Morales-Montor J. The role of cytokines in breast cancer development and progression. J Interferon Cytokine Res. 2015 January;35(1):1-16. doi: 10.1089/jir.2014.0026. Epub 2014 Jul 28. PMID: 25068787; PMCID: PMC4291218.
Fajgenbaum, D C and June, C H. 2020. Cytokine Storm. N Engl J Med. 383:2255-7.
Fasoulakis Z, Kolios G, Papamanolis V, Kontomanolis E N. Interleukins Associated with Breast Cancer. Cureus. 2018 Nov. 5; 10(11):e3549. doi: 10.7759/cureus.3549. PMID: 30648081; PMCID: PMC6324869.
Feghali, C A and Wright, T M. 1997. CYTOKINES IN ACUTE AND CHRONIC INFLAMMATION. Frontiers in Bioscience 2, d12-26.
Felman, A. (2021, October 28). Everything you need to know about preeclampsia. Medical News Today. https://www.medicalnewstoday.com/articles/252025
Fukata M et al (2007) Toll-like receptor-4 promotes the development of colitis-associated colorectal tumors. Gastroenterology 133: 1869-1881.
Harmon, A. C., Cornelius, D. C., Amaral, L. M., Faulkner, J. L., Cunningham, M. W., Jr, Wallace, K., & LaMarca, B. (2016). The role of inflammation in the pathology of preeclampsia. Clinical science (London, England: 1979), 130(6),409-419. https://doi.org/10.1042/CS20150702
Herrington, W, et al. 2016. Epidemiology of atherosclerosis and the potential to reduce the global burden of atherothrombotic disease. Circulation Research. 118(4): 535-546.
Hubel C. A. (1999). Oxidative stress in the pathogenesis of preeclampsia. Proceedings of the Society for Experimental Biology and Medicine. Society for Experimental Biology and Medicine (New York, N. Y.). 222(3), 222-235. https://doi.org/10.1046/j.1525-1373.1999.d01-139.x
Hunter, P. 2012, The Inflammation Theory of Disease. EMBO reports 13: 968-970.
Igyarto, B. Z., Jacobsen, S. & Ndeupen, S. 2021. Future considerations for the mRNA-lipid nanoparticle vaccine platform. Curr Opin Virol. 48:65-72.
Islam, M. O., Bacchetti, T. & Ferretti, G. 2019. Alterations of antioxidant enzymes and biomarkers of nitro-oxidative stress in tissues of bladder cancer. Oxid. Med. Cell Longev. 2730896 (2019).
Kaiser, J. (2021, April 22). Science. Science. https://www.science.org/content/article/clear-link-emerges-between-covid-19-and-pregnancy-complications
Kamaruzman N I, Aziz N A, Poh C L, Chowdhury E H. Oncogenic Signaling in Tumorigenesis and Applications of siRNA Nanotherapeutics in Breast Cancer. Cancers (Basel). 2019 May 6;11(5):632. doi: 10.3390/cancers11050632. PMID: 31064156; PMCID: PMC6562835.
Kaptoge, S, et al. 2014. Inflammatory cytokines and risk of coronary heart disease: new prospective study and updated meta-analysis. European Heart Journal. 35 (9):578-589.
Khalil, A., Samara, A., Chowdhury, T., & O'Brien, P. (2021, November). Does COVID-19 cause pre-eclampsia? Obstetrics & Gynecology. https://obgyn.onlinelibrary.wiley.com/doi/10.1002/uog.24809
Klampfer L. Cytokines, inflammation and colon cancer. Curr Cancer Drug Targets. 2011 May; 11(4):451-64. doi: 10.2174/156800911795538066. PMID: 21247378; PMCID: PMC3540985.
Kumari, N., Agrawal, U., Mishra, A. K., Kumar, A., Vasudeva, P., Mohanty, N. K., & Saxena, S. (2017). Predictive role of serum and urinary cytokines in invasion and recurrence of bladder cancer. Tumor Biology. https://doi.org/10.1177/1010428317697552
la Fuente, MD. 2002. Effects of antioxidants on immune system ageing European Journal of Clinical Nutrition. 56, Suppl 3, S5-S8.
Lee S J, Lee E J, Kim S K, Jeong P, Cho Y H, Yun S J, Kim S, Kim G Y, Choi Y H, Cha E J, Kim W J, Moon S K. Identification of pro-inflammatory cytokines associated with muscle invasive bladder cancer; the roles of IL-5, IL-20, and IL-28A. PLoS One. 2012;7(9):e40267. doi: 10.1371/journal.pone.0040267. Epub 2012 Sep 4. PMID: 22962576; PMCID: PMC3433484.
Li H, Chen C and Wang D W (2021) Inflammatory Cytokines, Immune Cells, and Organ Interactions in Heart Failure. Front. Physiol. 12:695047 doi: 10.3389/fphys.2021.695047
Li, R W S. et al. 2014. Uptake and protective effects of Ergothioneine in human endothelial cells. J Pharmacol. Exp.
Libby P, Ridker P M and Maseri A. 2002. Inflammation and atherosclerosis. Circulation 105(9):1135-1143.
Mann D L. Innate immunity and the failing heart: the cytokine hypothesis revisited. Circ Res. 2015 Mar. 27; 116(7):1254-68. doi: 10.1161/CIRCRESAHA.116.302317. PMID: 25814686; PMCID: PMC4380242.
Melina B. Pinheiro, Olindo A. Martins-Filho, Ana Paula L. Mota, Patricia N. Alpoim, Lara C. Godoi, Amanda C. O. Silveira, Andrea Teixeira-Carvalho, Karina B. Gomes, Luci M. Dusse, Severe preeclampsia goes along with a cytokine network disturbance towards a systemic inflammatory state, Cytokine, Volume 62, Issue 1, 2013, Pages 165-173.
Metz T D, et al. Association of SARS-CoV-2 infection with serious matemal morbidity and mortality from obstetric complications. Journal of the American Medical Association. 2022.
Monteleone, G, et al. 2020.Preventing Covid-19 induced pneumonia with anticytokine therapy. The Lancet. 2(5): E255-E256.
Morillon, A. C. et al. 2020. Effect of L-Ergothioneine on the metabolic plasma profile of the RUPP rat model of pre-eclampsia. PLOS ONE|https:/Idoi.org/10.1371/journal.pone.0230977.
Nash, S. D., Cruickshanks, K. J., Klein, R., Klein, B. E., Nieto, F. J., Chappell, R., Schubert, C. R., & Tsai, M. Y. (2013). Long-term variability of inflammatory markers and associated factors in a population-based cohort. Journal of the American Geriatrics Society, 61(8), 1269-1276. https://doi.orgi10.1111/jgs.12382
Natoli, G. et al. 2011. The genomic landscapes of inflammation. Genes & Dev. 25: 101-106
NHS website. (2021, November 18). Pre-eclampsia. Nhs.Uk. https://www.nhs.uk/conditions/pre-eclampsiai/#:%7E:text=There %20are %20a %20number%20of,pre %2Declampsia %20in %20a %20 previous
NIH-funded study suggests COVID-19 increases risk of pregnancy. (2022, February 7). National Institutes of Health (NIH). https://www.nih.gov/news-events/news-releases/nih-funded-study-suggests-covid-19-increases-risk-pregnancy-complications
Pahwa R, Goyal A, Jialal I. Chronic Inflammation. [Updated 2022 Jun 19]. In: StatPearls [Internet]. Treasure Island (FL): StatPearls Publishing; 2022 Jan-. Available from: 30 https://www.ncbi.nlm.nih.gov/books/NBK493173/
Phaniendra, A., Jestadi, D. B.& Periyasamy, L. 2015. Free radicals: Properties, sources, targets, and their implication in various diseases. Indian J. Clin. Biochem. 30(1): 11-26.
Prabhu, S. (2004). Cytokine-Induced Modulation of Cardiac Function. Circulation Research. Retrieved Sep. 5, 2022, from https://www.ahajoumals.org/doi/10.1161/01.res.0000150734.79804.92
Riley, L. (2021, June). mRNA Covid-19 Vaccines in Pregnant Women. Https://Www.Nejm.Org/Doi/Full/10.1056/NEJMe2107070. https://www.nejm.org/doi/full/10.1056/NEJMe2107070
Sawicka, E. et al. 2020. Preliminary Study on Selected Markers of Oxidative Stress, Inflammation and Angiogenesis in Patients with Bladder Cancer. Pathology & Oncology Research 26:821-831
Shu C, Liu Z, Cui L, Wei C, Wang S, Tang J J, Cui M, Lian G, Li W, Liu X, Xu H, Jiang J, Lee P, Zhang D Y, He J, Ye F. Protein profiling of preeclampsia placental tissues. PLoS One. 2014 Nov. 13; 9(11):e112890. doi: 10.1371/journal.pone.0112890. PMID: 25392996; PMCID: PMC4231077.
Smith, R. 2004. “Let Food Be Thy Medicine . . . ” BMJ. 328:7433
Srinivasan, K. et al. 2016. Untangling the brain's neuroinflammatory and neurodegenerative transcriptional responses. Nature Communications. DOI: 10.1038.
Stefánska K, Zieliński M, Jankowiak M, Zamkowska D, Sakowska J, Adamski P, Jassem-Bobowicz J, Piekarska K, Leszczynska K, Świtkowska-Stodulska R, Kwiatkowski S, Preis K, Trzonkowski P and Marek-Trzonkowska N (2021) Cytokine Imprint in Preeclampsia. Front. Immunol. 12:667841. doi: 10.3389/fimmu.2021.667841
Sun, X et al. 2020. Cytokine storm intervention in the early stages of COVID-19 pneumonia. Cytokine Growth Factor Rev. 53: 38-42
Tenenbaum J D. 2016. Translational Bioinformatics: Past, Present, and Future. Genomics Proteomics Bioinformatics. 14(1): 31-41.
Turner, E. et al. 2009. Imidazole-based erythrocyte markers of oxidative stress in preeclampsia—an NMR investigation. Reprod Sci. 16(11):1040-51.
Uttara, B et al. 2009. Oxidative Stress and Neurodegenerative Diseases: A Review of Upstream and Downstream Antioxidant Therapeutic Options. Current Neuropharmacology, 2009, 7, 65-74.
Weaver, C M and Miller, J W. 2017. Challenges in conducting clinical nutrition research. Nutrition Reviews. 75(7):491-499.
Wigner, P. et al. 2021. Oxidative stress parameters as biomarkers of bladder cancer development and progression. Scientific Reports, Nature 11:15134.
Williamson, R., McCarthy, F., Manna, S., Groarke, E., Kell, D., Kenny, L., & McCarthy, C. (2020, February). L-(+)-Ergothioneine Significantly Improves the Clinical Characteristics of S Preeclampsia in the Reduced Uterine Perfusion Pressure Rat Model. American Heart Association. https://www.ahajoumals.org/doi/epub/10.1161/HYPERTENSIONAHA.119.13929
Wu, J., & Wu, L. (2007). Acute and Chronic Inflammation: Effect of the Risk Factor (s) on the Progression of the Early Inflammatory Response to the Oxidative and Nitrosative Stress. Lab Med. https://www.labmed.org.tw/upfiles/issues/20091221155246.pdf
Wu, M Y, et al. 2017. New Insights into the Role of Inflammation in the Pathogenesis of Atherosclerosis. Int. J. Mol. Sci. 18: 2034
Zhang, M. 2022. Inflammatory mRNA Nanoparticles Inhibit and Alter Immune Response. Time. Aug. 31, 2022.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A method of determining whether a patient has bladder cancer, colon cancer, or breast cancer, the method comprising
a) measuring an mRNA level of at least one gene in a tissue of the patient, wherein the at least one gene is KRT1, CCL27, SDK1, PPARG, IL1A, SMAD3, SLC6A3, NR3C1, KRT83, HFE2, CASP3, CACNA1C, NFKB2, CASP9, or any combination thereof;
b) comparing the mRNA levels determined in a) with an mRNA level of the at least one gene from a reference tissue from
i) at least one subject with bladder cancer, and/or ii) at least one subject with colon cancer, and/or iii) at least one subject with breast cancer,
wherein A) the patient has bladder cancer if the mRNA level(s) determined in a) is similar to the mRNA level from b)i); the patient has colon cancer if the mRNA level(s) determined in a) is similar to the mRNA level from b)ii); and the patient has breast cancer if the mRNA level(s) determined in a) is similar to the mRNA level from b)iii).

2. The method of claim 1, wherein the at least one gene is KRT1, CCL27, SDK1, PPARG or any combination thereof, and the method determines whether the patient has breast cancer or colon cancer.

3. The method of claim 2, wherein the at least one gene is KRT1, CCL27 or the combination thereof.

4. The method of claim 1, wherein the at least one gene is IL1A, SMAD3 or the combination thereof, and the method determines whether the patient has bladder cancer or colon cancer.

5. The method of claim 1, wherein the at least one gene is IL1A, CCL27 or the combination thereof, and the method determines whether the patient has bladder cancer or breast cancer.

6. The method of claim 1, wherein the at least one gene is SLC6A3, NR3C1, KRT1, KRT83, HFE2, CCL27, IL1A, SMAD3, CASP3, SDK1, CACNA1C, NFKB2, PPARG, CASP9, or any combination thereof, and the method determines whether the patient has bladder cancer, breast cancer or colon cancer.

7. The method of claim 6, wherein the at least one gene is SLC6A3, NR3C1, KRT1, KRT83, HFE2, CCL27, or any combination thereof.

8. The method of claim 1, The method of claim 1, wherein the tissue is a saliva, blood, cell or urine sample.

9. The method of any one of claims 1-8, further comprising recommending a treatment for bladder cancer if the mRNA level(s) determined in a) is similar to the mRNA level from b)i); recommending a treatment for colon cancer if the mRNA level(s) determined in a) is similar to the mRNA level from b)ii); or recommending a treatment for breast cancer if the mRNA level(s) determined in a) is similar to the mRNA level from b)iii).

10. The method of any one of claims 1-8, further comprising treating the patient for bladder cancer if the mRNA level(s) determined in a) is similar to the mRNA level from b)i); treating the patient for colon cancer if the mRNA level(s) determined in a) is similar to the mRNA level from b)ii); or treating the patient for breast cancer if the mRNA level(s) determined in a) is similar to the mRNA level from b)iii).

11. A method of determining whether a patient has a chronic inflammatory disease, the method comprising
determining expression levels in the patient of one or more genes or protein biomarkers listed in Table 1 or measurement of 80 HdG levels, wherein
(a) if the expression levels indicate inflammation according to Table 1 or measurement of 80 HdG levels, the patient has a chronic inflammatory disease, or
(b) if the expression levels do not indicate inflammation according to Table 1 or measurement of 80 HdG levels, the patient does not have a chronic inflammatory disease.

12. The method of claim 11, wherein
(a) if the expression levels indicate inflammation according to Table 1 or measurement of 80 HdG levels, recommending that the patient undergo a treatment indicated in Table 1 under “Panel Results Indicate Inflammation” and
(b) if the expression levels do not indicate inflammation according to Table 1 or measurement of 80 HdG levels, recommending that the patient undergo treatment indicated in Table 1 under “Panel Results Do Not Indicate Inflammation”.

13. The method of claim 11, wherein
(a) if the expression levels indicate inflammation according to Table 1 or measurement of 80 HdG levels, treating the patient with a treatment that reduces inflammation and
(b) if the expression levels do not indicate inflammation according to Table 1 or measurement of 80 HdG levels, treating the patient with a treatment that does not reduce inflammation.

14. The method of claim 11, wherein the expression levels are determined by determining mRNA levels of the one or more genes or protein biomarkers in blood, urine, cells, or a tissue of the patient.

15. The method of claim 14, wherein the one or more genes or protein biomarkers are ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, GF1, IL-1, IL-10, IL-18, IL-1B, IL-6, KRT1, KRT83, STK11, MMP13, MUC1, NFKB1, NFKB2, NLRP3, NLRP1, NR3C1, PAX8, PDL1, PPARG, RGS2, SDK1, SIRT1, SKA2, SLC11A2, SLC22A4, SLC40A1, SLC6A3, SMAD3, TGFB1, TNF, VEGFA, IL-8, IL-17, TNF-α, TGF-β1, CASP-8, CASP-10, CASP-1, TRAIL, 80 HdG or any combination thereof.

16. The method of claim 11, wherein the expression levels are determined by determining levels of the protein encoded by the one or more genes or protein biomarkers in blood, urine, saliva, cells or a tissue of the patient.

17. The method of claim 16, wherein the tissue where levels of the protein encoded by the one or more genes or protein biomarkers is determined is a tissue that exhibits signs of the disease.

18. The method of claim 16, wherein the protein biomarker is Interleukin1 (IL-1), Interleukin-6 (IL-6), Interleukin-8 (IL-8), Interleukin-10 (IL-10), Interleukin-17 (IL-17), Interleukin-18 (IL-18), Tumor necrosis factor-α (TNF-α), Transforming growth factor-β (TGF-β1), Caspase 8, Caspase 10, Caspase 1, TRAIL, 80 HdG or any combination thereof.

19. The method of any one of claims 11-18, wherein the disease is preeclampsia, bladder cancer, breast cancer, colon cancer, or a cardiac disease.

20. The method of claim 19, wherein the disease is also diagnosed, monitored or prognosed by determining whether genes are up regulated or down regulated in accordance with Table 1.

21. The method of claim 16, wherein the disease is preeclampsia.

22. The method of claim 16, wherein the disease is bladder cancer.

23. The method of claim 16, wherein the disease is breast cancer.

24. The method of claim 16, wherein the disease is colon cancer.

25. The method of claim 16, wherein the disease is a cardiac disease.

26. The method of claim 25, wherein the cardiac disease is atherosclerosis or vasculitis.

27. A method of evaluating patient response to a treatment for a chronic inflammatory disease, the method comprising
a) determining expression of at least one or more genes or protein biomarkers listed in Table 1 or measurement of 80 HdG levels in a population of subjects without the chronic inflammatory disease;
b) determining expression in the patient of the at least one or more genes or protein biomarkers listed in Table 1 or measurement of 80 HdG levels prior to the treatment; and
c) determining expression of the at least one or more genes or protein biomarkers after the treatment,
wherein i) the patient is responding to the treatment if expression of the at least one or more genes or protein biomarkers approaches the expression in a) after treatment, and
ii) the patient is not responding to the treatment if expression of the at least one or more genes or protein biomarkers does not approach the expression in a) after treatment.

28. The method of claim 27, wherein the one or more genes or protein biomarkers are ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, GF1, IL-1, IL-10, IL-18, IL-1B, IL-6, KRT1, KRT83, STK11, MMP13, MUC1, NFKB1, NFKB2, NLRP3, NLRP1, NR3C1, PAX8, PDL1, PPARG, RGS2, SDK1, SIRT1, SKA2, SLC11A2, SLC22A4, SLC40A1, SLC6A3, SMAD3, TGFB1, TNF, VEGFA, IL-8, IL-17, TNF-α, TGF-β1, CASP-8, CASP-10, CASP-1, TRAIL, 80 HdG or any combination thereof.

29. The method of claim 27, wherein the disease is preeclampsia, bladder cancer, breast cancer, colon cancer, or a cardiac disease.

30. The method of claim 27, wherein the disease is bladder cancer, breast cancer, or colon cancer and the one or more genes or protein biomarkers are KRT1, CCL27, SDK1, PPARG, IL1A, SMAD3, SLC6A3, NR3C1, KRT83, HFE2, CASP3, CACNA1C, NFKB2, CASP9, or any combination thereof.

31. The method of claim 30, wherein the one or more genes or protein biomarkers are SLC6A3, NR3C1, KRT1, KRT83, HFE2, CCL27, or any combination thereof.

32. The method of claim 27, wherein the disease is breast cancer or colon cancer and the one or more genes or protein biomarkers are KRT1, CCL27, SDK1, PPARG or any combination thereof.

33. The method of claim 32, wherein the one or more genes or protein biomarkers are KRT1, CCL27 or the combination thereof.

34. The method of claim 27, wherein the disease is bladder cancer or colon cancer and the one or more genes or protein biomarkers are ILIA, SMAD3 or the combination thereof.

35. The method of claim 27, wherein the disease is bladder cancer or breast cancer and the one or more genes or protein biomarkers are ILIA, CCL27 or the combination thereof.

37. The method of any one of claims 27-35, further comprising recommending no change in treatment if i), or recommending a change in treatment if ii).

38. The method of any one of claims 27-35, further comprising maintaining the treatment if i), or changing the treatment if ii).

39. A method of developing a treatment for a chronic inflammatory disease in a patient, the method comprising
(a) obtaining a potential treatment medication;
(b) determining expression levels in the patient of one or more genes or protein biomarkers listed in Table 1 or measurement of 80 HdG levels prior to administering the medication;
(c) administering the medication to the patient;
(d) determining expression levels in the patient of the one or more genes or protein biomarkers after administering the medication; and
(e) establishing the medication as a treatment for the disease if a change in expression levels between step (b) and step (d) indicates that the medication reduces or increases inflammation.

40. The method of claim 39, wherein the chronic inflammatory disease is preeclampsia, bladder cancer, breast cancer, colon cancer, or a cardiac disease.

41. The method of claim 39 or 40, wherein the expression levels are determined by determining mRNA levels of the one or more genes or protein biomarkers in blood, urine, saliva, cells, or a tissue of the patient.

42. The method of claim 41, wherein the one or more genes or protein biomarkers are ABCA1, ADAMTS4, CACNA1C, CASP1, CASP3, CASP9, CCL27, CRHR1, EDAR, EPO, FGFR2, FKBP5, GCKR, GH1, HAMP, HFE, HFE2, GF1, IL-1, IL-10, IL-18, IL-1B, IL-6, KRT1, KRT83, STK11, MMP13, MUC1, NFKB1, NFKB2, NLRP3, NLRP1, NR3C1, PAX8, PDL1, PPARG, RGS2, SDK1, SIRT1, SKA2, SLC11A2, SLC22A4, SLC40A1, SLC6A3, SMAD3, TGFB1, TNF, VEGFA, IL-8, IL-17, TNF-α, TGF-β1, CASP-8, CASP-10, CASP-1, TRAIL, 80 HdG or any combination thereof.

43. The method of claim 39 or 40, wherein the expression levels are determined by determining levels of the protein encoded by the one or more genes or protein biomarkers in a tissue of the patient.

44. The method of claim 43, wherein the tissue where levels of the protein encoded by the one or more genes or protein biomarkers is determined is a tissue that exhibits signs of the disease.

45. The method of claim 43 or 44, wherein the genes or protein biomarkers is Interleukin1 (IL-1), Interleukin-6 (IL-6), Interleukin-8 (IL-8), Interleukin-10 (IL-10), Interleukin-17 (IL-17), Interleukin-18 (IL-18), Tumor necrosis factor-α (TNF-α), Transforming growth factor-β (TGF-β1), Caspase 8, Caspase 10, Caspase 1, TRAIL, 80 HdG or any combination thereof.
